(19) United States
(12) Patent Application Publication
(10) Pub. No.: US 2012/0196887 A1
(43) Pub. Date: Aug. 2, 2012

(54) METHODS AND COMPOSITIONS FOR IMPROVED RIFAMYCIN THERAPIES

(76) Inventors: Charles DARKOH, Houston, TX (US); Herbert L. DUPONT, Houston, TX (US); Lenard M. LICHTENBERGER, Houston, TX (US); Elizabeth J. Dial, Houston, TX (US)

(21) Appl. No.: 13/364,589

(22) Filed: Feb. 2, 2012

Related U.S. Application Data
(60) Provisional application No. 61/438,777, filed on Feb. 2, 2011.

(51) Int. Cl. A61K 31/437; A61P 31/00; A61P 1/00; B82Y 5/00

(52) U.S. Cl. 514/279; 977/788; 977/915

(57) ABSTRACT

Compositions comprising one or more rifamycin antibiotics and one or more bile acids, and methods of using the compositions for the treatment of infection.

### CROSS-REFERENCE TO RELATED APPLICATIONS

The present application claims the benefit of U.S. Provisional Application Ser. No. 61/438,777 filed Feb. 2, 2011, which is herein incorporated by reference in its entireties for all purposes.

### STATEMENT REGARDING FEDERALLY SPONSORED RESEARCH OR DEVELOPMENT

This invention was made with U.S. Government support under Grant No. DK56338 awarded by the National Institutes of Health. The government has certain rights in the invention.

### TECHNICAL FIELD

This disclosure relates to methods and compositions which can be used to increase the solubility and bioavailability of antibiotics for use in treatment of infection. More particularly, this disclosure relates to the use of such methods and compositions in increasing the solubility and bioavailability of poorly soluble antibiotics, particularly derivatives of the rifamycin class of antibiotics (e.g., a pyrido-imidazo rifamycin), which includes rifampicin (rifampin), rifabutin and rifapentine and derivatives thereof. Among other things, increasing solubility, increases the active concentration of antibiotic in solution and thus allows one to administer reduced dosages, which increase safety margins as well as decreasing the overall cost of therapies involving the use of antibiotics.

### BACKGROUND

The rifamycin class of antibiotics was originally isolated from cultures of Streptomyces mediterranei. Eventually multiple rifamycins were discovered (rifamycin A, B, C, D, E, S and SV) and rifamycin B was introduced commercially (see patent Nos: GB921045 and U.S. Pat. No. 3,150,046) and is the precursor of various other clinically-utilized potent derivatives. The mechanism of action of rifamycins is believed to lie in the inhibition of DNA-dependent RNA synthesis as the result of high affinity binding of prokaryotic RNA polymerase by rifamycins and a resulting steric inhibition of oligonucleotide chain elongation. Due to the large number of available analogues and derivatives generated synthetically, rifamycins have been widely utilized in the elimination of pathogenic bacteria that have become resistant to commonly used antibiotics. Currently marketed examples of the rifamycin class of antibiotics include, for example, rifampicin (rifampin), rifabutin, rifapentine and rifaximin.

Rifampicin (CAS number 13292-46-1) also known as rifaldazine, R/AMP, rofact (in Canada), and rifampin in the U.S. is a semisynthetic bactericidal antibiotic drug derived from various types of rifamycins. Rifampicin is sold by different names worldwide, alone and in combination with isoniazid and pyrazinamide (TUBOCIN, SINERDOL, RIFADIN, RIMACTAN, RIFATER, RIFINAH, RIMACTAZID, RIMACTANE, RIFADINE, R-CINEX 600, and RIMYCIN) and used for the treatment of many diseases, most importantly tuberculosis acquired in HIV-positive patients. Rifampicin is associated with a range of adverse effects, including hepatotoxicity, in part because it induces upregulation of hepatic cytochrome P450 enzymes (such as CYP2C9 and CYP3A4) and therefore, also affects the rate of metabolism of other drugs that are cleared by the liver.

Rifabutin (CAS Number 72559-06-9) is a semi-synthetic derivative of rifamycin S. It is sold under the brand name MYCOBUTIN and is effective against Gram-positive and some Gram-negative bacteria, but also against the highly resistant Mycobacteria, e.g. Mycobacterium tuberculosis, M. leprae and M. avium intracellulare and Chlamydophila pneumonia and is therefore used in the treatment of tuberculosis and Chlamydia, particularly in AIDS patients.

Rifapentine (CAS number 61379-65-5) is an antibiotic synthesized from rifampicine and is marketed under the brand name PRIFTIN and is also used in the treatment of tuberculosis.

In contrast to the more soluble and absorbed rifamycin class antibiotic compounds described above, rifaximin (CAS number 80621-81-4) is a semisynthetic, rifamycin-based non-systemic antibiotic, that is largely water-insoluble, non-absorbable (<0.4%) antibiotic that inhibits bacterial RNA synthesis. Rifaximin is licensed by the U.S. Food and Drug Administration to treat traveler's diarrhea (and hepatic encephalopathy for which it received orphan drug status) caused by diarrhea producing E. coli and clinical trials have shown that rifaximin is highly effective at preventing and treating traveler's diarrhea among travelers to Mexico. Rifaximin in not currently believed to be effective against Campylobacter jejuni, and there is no recognized efficacy against Shigella or Salmonella species. Rifaximin may also be efficacious in relieving chronic functional symptoms of bloating and flatulence that are common in irritable bowel syndrome (see for example, Sharara A, et al., A randomized double-blind placebo-controlled trial of rifaximin in patients with abdominal bloating and flatulence. Am J Gastroenterol 101 (2): 326, 2006). Rifaximin is currently available in the U.S. under the brand name Xifaxan by Salix Pharmaceuticals. It is also sold in Europe under the names Spiraxin, Zaxine, Normix, Rifacol and Colidur and in India-tinder the name RIXMIN. However it is expensive and as many of the cases of diarrhea occur in underdeveloped countries, consequently, there is continuing interest in reducing the cost of therapy and one way to do this is to increase the solubility/bioavailablity of the active compound.

Rifaximin is well tolerated and does not appear to induce significant levels of resistance in enteric flora during repeated dosing and rifaximin also has minimal effects on colonic flora (DuPont, H. L., and Z. D. Jiang. Influence of rifaximin treatment on the susceptibility of intestinal Gram-negative flora and enterococci. Clin Microbiol Infect. 10:1009-11, 2004; DuPont, H. L., et al. A randomized, double-blind, placebo-controlled trial of rifaximin to prevent travelers' diarrhea. Ann Intern Med 142:805-12, 2005). In the U.S. rifaximin has orphan drug status for the treatment of hepatic encephalopathy. Rifaximin is currently sold in the U.S. under the brand name XIFAXAN by Salix Pharmaceuticals. It is also sold in Europe under the names SPIRAXIN, ZAXINE, NORMIX, RIFACOL and COLIDUR and in India as RIXMIN.

A similar compound, Rifamycin SV has been formulated using Multi Matrix (MMX®) to create Rifamycin SV MMX® (Santarus, Inc. in a strategic collaboration with Cosmo Pharmaceuticals S.p.A. (Lainate, Italy) in which coating with pH-resistant acrylic copolymers delays the release of the Rifamycin SV until the tablet reaches the indicated intestinal location where the programmed dissolution begins. This facilitates delivery into the lumen of the colon and provides controlled release along the length of the colon. The specific dissolution profile of Rifamycin SV MMX® tablets is thought to increase the colonic disposition of the antibiotic so that an optimized intestinal concentration is achieved thus reducing its early inactivation by metabolic reactions or dilution and thus abating its systemic absorption in the small intestine.

Bowel or gastrointestinal disorders are not only uncomfortable, but can be debilitating and even fatal. For example, diarrhea is one of the most common infirmities affecting international travelers; occurring in 20-50% of persons visiting developing regions from industrialized countries. Traveler's diarrhea is defined as three or more unformed stools in 24 hours passed by a traveler, commonly accompanied by abdominal cramps, nausea, and bloating. Infectious agents are the primary cause of travelers' diarrhea.

Bacterial enteropathogens cause approximately 80% of all cases. Enterotoxigenic Escherichia coli (ETEC) is the most common causative agent isolated in approximately half (ranging from 20-75%) of the cases of travelers' diarrhea. ETEC produces two notable enterotoxins, a cholera-like heat-labile (LT) and small molecular weight heat-stable (ST) toxin. ETEC is also important cause of pediatric diarrhea and death in developing countries. The diarrhea-producing E. coli important in travelers' diarrhea are ETEC and enteroaggregative E. coli (EAEC) (Jiang, Z. D., et al. Prevalence of enteric pathogens among international travelers with diarrhea acquired in Kenya (Mombasa), India (Goa), or Jamaica (Montego Bay). J Infect Dis. 185:497-502, 2002). Both ETEC and EAEC are known to be small bowel pathogens. Other bacterial pathogens that cause gastrointestinal disorders and diarrhea include: Shigella species (2-30%) and Salmonella species (0-33%) as well as for example, Campylobacter, Yersinia, Aeromonas, and Plesiomonas species can also be the cause of gastrointestinal distress and diarrhea. There is a continuing need for more effective antibiotic compositions for treatment of bowel or gastrointestinal disorders.

### SUMMARY

The presently disclosed compositions and methods are based, in part, on the discovery that rifaximin is significantly more soluble in solutions comprising one or more bile acids than in aqueous solution. The examples set forth herein demonstrate that the addition of both purified bile acids and human bile to rifaximin at sub-inhibitory and inhibitory concentrations significantly improved the drug's activity against enteric disorders and pathogens such as, but not limited to, ETEC, Enteroaggregative E. coli, Shigella flexneri and Salmonella enteric. For example, the anti-ETEC activity was increased by greater than 70% after 4 hours. The data demonstrate that bile acids solubilize rifaximin in a dose dependent fashion resulting in an increase in the drug's bioavailability and antimicrobial effect.

Accordingly, the instant application sets forth compositions and methods for increasing the efficacy of rifamycins and in particular rifaximin, while decreasing the required dosage and thus potential toxicities. The exemplary results of associating bile salts with rifaximin are considered representative of those achieved with other rifamycin compounds. Thus, it is proposed that association with bile acids also increases the solubility of other members of the rifamycin group of antibiotics such as rifampicin (rifampin), rifabutin, rifapentine, rifaximin and poorly soluble derivatives thereof. In many embodiments, increased solubility of rifamycin antibiotic compounds will reduce the amount of compound needed to treat a disorder and may also reduce the expense associated with treating the disorder. Also, by reducing the amount of compound needed one also reduces the potential toxicity of a therapeutic regimen.

In accordance with certain embodiments, a composition is provided comprising a rifamycin compound and at least one bile acid. For example, the rifamycin compound is rifaximin or a polymorphic form of rifaximin in some cases.

In some embodiments of an above-described composition the bile acid is cholic acid, chenodeoxycholic acid, deoxycholic acid, lithocholic acid, glycocholic acid, taurocholic acid, glycocholic acid, glycodeoxycholic acid, glycochenodeoxycholic acid, taurocholic acid and taurodeoxycholic acid, glycolithocholic acid, taurolithocholic acid, taurohyodeoxycholic acid, taurochenodeoxycholic acid, ursocholic acid, tauroursodeoxycholic acid or glycoursodeoxycholic acid. In some embodiments, the bile acid is human bile acid, and in certain embodiments the bile acid is synthetic bile acid.

In some embodiments of an above-described composition, a bile acid forms mixed micelles in water. In some embodiments, an above-described composition comprises nanoparticles containing a rifamycin compound and one or more bile acids. In many embodiments of an above-described composition, the composition is aqueous and the solubility of the rifaximin in the aqueous composition is enhanced compared to a like composition lacking the bile acid component.

In accordance with other embodiments, a pharmaceutical composition is provided which comprises an above-described composition and a pharmaceutically acceptable carrier.

In accordance with other embodiments, a method of treating an infection of the gastrointestinal tract or colon in a subject is provided which comprises administering to the subject an above-described composition to treat the infection.

In accordance with other embodiments, a method for increasing the efficacy of rifaximin treatment of a gastrointestinal disorder in a subject is provided which comprises administering to the subject an above-described composition, thereby increasing the efficacy of an amount of rifaximin for treating the disorder as compared to treatment of the disorder in the subject with the same amount of rifaximin in the absence of the bile acid.

A method for increasing the solubility of rifaximin in aqueous solution is provided in accordance with still another embodiment, and comprises formulating an aqueous composition comprising an amount of rifaximin and at least one bile acid, wherein the solubility of the amount of rifaximin in the aqueous composition is greater than the solubility of the same amount of rifaximin in an aqueous composition lacking the bile acid.

In accordance with still another embodiment, a kit is provided which comprises an above-described composition, or pharmaceutical composition, and instructions for use.

These and other embodiments will be apparent in the following description.

### BRIEF DESCRIPTION OF THE DRAWINGS

FIG. 1 depicts the solubility of rifaximin (12 mg) in water and equimolar concentrations of bile acids: cholic, chenodeoxycholic, deoxycholic, sodium glycocholate, lithocholic, and taurocholic acids in a mixture at pH 7.4. Total bile acids concentration at each reading=Individual bile acid concentration multiplied by 6.

FIG. 2 depicts the growth of ETEC Strain H10407 in the presence of 16 μg/mL rifaximin in water and 4 mM human bile acid at pH 7.4. Cells were grown with rifaximin in the presence and absence of human bile and absorbance at 600 nm was measured at 30 minutes intervals. Mann-Whitney two-tailed non-parametric t-test analysis showed a statistically significant difference between treatments: No rifaximin+No bile acids vs. 16 μg/mL Rifaximin (p=0.026, n=4); No rifaximin+No bile acids vs. 16 μg/mL rifaximin+bile acids (p=0.002, n=4); 16 μg/mL rifaximin vs. 16 μg/mL rifaximin+bile acids (p=0.012, n=4). The error bars represent the standard deviation between four replicate experiments.

FIG. 3 depicts the growth of ETEC Strain H10407 in the presence of rifaximin (16 μg/mL) in water and equimolar mixture of synthetic bile acids: cholic, deoxycholic, chenodeoxycholic, glycocholic, lithocholic, and taurocholic acids at pH 7.4. Total bile acids concentration=4 mM. Cells were grown with rifaximin in the presence and absence of bile acids and absorbance at 600 nm was measured at 30 minutes intervals. Mann-Whitney two-tailed non-parametric t-test analysis showed a statistically significant difference between treatments: No rifaximin+No bile acid Mann-Whitney two-tailed non-parametric t-test ids vs. 16 μg/mL Rifaximin (p=0.026, n=4); No rifaximin+No bile acids vs. 16 μg/mL rifaximin+bile acids (p=0.002, n=4); 16 μg/mL rifaximin vs. 16 μg/mL rifaximin+bile acids (p=0.007, n=4). The error bars represent the standard deviation between four replicate experiments.

FIG. 4 depicts the growth of ETEC Strain H10407 after 4 hours incubation at 37° C. in the presence of rifaximin (8 μg/mL, 16 μg/mL, and 32 μg/mL) in water and 4 mM total synthetic bile acids in a pooled mixture: cholic, deoxycholic, chenodeoxycholic, glycocholic, lithocholic and taurocholic acids at pH 7.4. The error bars represent the standard deviation between four replicate experiments.

FIG. 5 depicts the effect of bile acids on the activity of β-galactosidase enzyme. Each treatment contained 4 mM of total synthetic bile acids in a mixture: cholic, deoxycholic, chenodeoxycholic, glycocholic, lithocholic and taurocholic acids at pH 7.4. One unit=Amount of enzyme required to convert a micromole of O-nitrophenyl-β-D-galactoside to O-nitrophenol and galactose per minute at pH 7.4 at 30° C. The error bars represent the standard deviation between three replicate experiments.

FIG. 6 depicts the evaluation of total protein content of bacteria treated with 16 μg/mL rifaximin and bile acids. Each treatment contained 4 mM of total synthetic bile acids in a mixture: cholic, deoxycholic, chenodeoxycholic, glycocholic, lithocholic and taurocholic acids at pH 7.4. Cells were grown with rifaximin in the presence and absence of bile acids and aliquots were taken every hour for total protein determination. Total protein concentration was determined using Bradford assay. The error bars represent the standard deviation between three replicate experiments.

FIG. 7 depicts the growth of ETEC Strain H10407 in the presence of rifaximin (16 μg/mL) in water and 4 mM of the single synthetic bile acid: cholic at pH 7.4. Cells were grown with rifaximin in the presence and absence of bile acids and absorbance at 600 nm was measured at 30 minutes intervals.

FIG. 8 depicts the growth of ETEC Strain H10407 in the presence of rifaximin (16 μg/mL) in water and 4 mM of the single synthetic bile acid: deoxycholic at pH 7.4. Cells were grown with rifaximin in the presence and absence of bile acids and absorbance at 600 nm was measured at 30 minutes intervals.

FIG. 9 depicts the growth of ETEC Strain H10407 in the presence of rifaximin (16 μg/mL) in water and 4 mM of the single synthetic bile acid: chenodeoxycholic at pH 7.4. Cells were grown with rifaximin in the presence and absence of bile acids and absorbance at 600 nm was measured at 30 minutes intervals.

FIG. 10 depicts the growth of ETEC Strain H10407 in the presence of rifaximin (16 μg/mL) in water and 4 mM of the single synthetic bile acid: glycocholic at pH 7.4. Cells were grown with rifaximin in the presence and absence of bile acids and absorbance at 600 nm was measured at 30 minutes intervals.

FIG. 11 depicts the growth of ETEC Strain H10407 in the presence of rifaximin (16 μg/mL) in water and 4 mM of the single synthetic bile acid: lithocholic at pH 7.4. Cells were grown with rifaximin in the presence and absence of bile acids and absorbance at 600 nm was measured at 30 minutes intervals.

FIG. 12 depicts the growth of ETEC Strain H10407 in the presence of rifaximin (16 μg/mL) in water and 4 mM of the single synthetic bile acid: taurocholic at pH 7.4. Cells were grown with rifaximin in the presence and absence of bile acids and absorbance at 600 nm was measured at 30 minutes intervals.

FIG. 13 depicts the growth of Shigella flexneri (serotype 2B) in the presence of rifaximin (32 μg/mL) in water and equimolar mixture of synthetic bile acids: cholic, deoxycholic, chenodeoxycholic, glycocholic, lithocholic, and taurocholic acids at pH 7.4. Total bile acids concentration=4 mM. Cells were grown under the conditions shown absorbance at 600 nm was measured every hour. RIFA=Rifaximin. The minimal inhibitory concentration (MIC) of this strain for rifaximin is 64 μg/mL.

FIG. 14 depicts the total protein amount of Shigella flexneri treated with 32 μg/mL rifaximin and bile acids. Each treatment contained 4 mM of total synthetic bile acids in a mixture: cholic, deoxycholic, chenodeoxycholic, glycocholic, lithocholic and taurocholic acids at pH 7.4. Cells were grown under the conditions shown and aliquots were taken every two hours for total protein determination using Bradford protein assay.

FIG. 15 depicts the growth of Salmonella enterica (ATCC #14028) in the presence of rifaximin (32 μg/mL) in water and equimolar mixture of synthetic bile acids: cholic, deoxycholic, chenodeoxycholic, glycocholic, lithocholic, and taurocholic acids at pH 7.4. Total bile acids concentration=4 mM. Cells were grown under the conditions shown absorbance at 600 nm was measured every hour. RIFA=Rifaximin. The MIC of this strain for rifaximin is 64 μg/mL.

FIG. 16 depicts the total protein amount of Salmonella enterica treated with 32 μg/mL rifaximin and bile acids. Each treatment contained 4 mM of total synthetic bile acids in a mixture: cholic, deoxycholic, chenodeoxycholic, glycocholic, lithocholic and taurocholic acids at pH 7.4. Cells were grown under the conditions shown and aliquots were taken every two hours for total protein determination using Bradford protein assay.

FIG. 17 depicts the growth of Enteroaggregative E. coli in the presence of rifaximin (16 μg/mL) in water and equimolar mixture of synthetic bile acids: cholic, deoxycholic, chenodeoxycholic, glycocholic, lithocholic, and taurocholic acids at pH 7.4. Total bile acids concentration=4 mM. Cells were grown under the conditions shown absorbance at 600 nm was measured every hour. RIFA=Rifaximin. The MIC of this strain for rifaximin is 32 μg/mL.

FIG. 18 depicts the total protein amount of enteroaggregative E. coli treated with 16 μg/mL rifaximin and bile acids. Each treatment contained 4 mM of total synthetic bile acids in a mixture: cholic, deoxycholic, chenodeoxycholic, glycocholic, lithocholic and taurocholic acids at pH 7.4. Cells were grown under the conditions shown and aliquots were taken every two hours for total protein determination using Bradford protein assay.

### DETAILED DESCRIPTION

### Definitions

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components, that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, and unless otherwise indicated, the terms “treat,” “treating,” “treatment” and “therapy” contemplate an action that occurs while a patient is suffering from a rifamycin sensitive disorder that reduces the severity of one or more symptoms or effects of the rifamycin sensitive disorder, such as but not limited to bowel or gastrointestinal disorder or a related disease or disorder. Where the context allows, the terms “treat,” “treating,” and “treatment” also refers to actions taken toward ensuring that individuals at increased risk of a rifamycin sensitive disorder, such as but not limited to bowel or gastrointestinal disorder are able to receive appropriate surgical and/or other medical intervention prior to onset of a rifamycin sensitive disorders, such as but not limited to bowel or gastrointestinal disorders. As used herein, and unless otherwise indicated, the terms “prevent,” “preventing,” and “prevention” contemplate an action that occurs before a patient begins to suffer from rifamycin sensitive disorder, such as but not limited to bowel or gastrointestinal disorder, that delays the onset of, and/or inhibits or reduces the severity of, a rifamycin sensitive disorder, such as but not limited to bowel or gastrointestinal disorder.

As used herein, and unless otherwise indicated, the terms “manage,” “managing,” and “management” encompass preventing, delaying, or reducing the severity of a recurrence of rifamycin sensitive disorders, such as but not limited to bowel or gastrointestinal disorders in a patient who has already suffered from such a disease, disorder or condition. The terms encompass modulating the threshold, development, and/or duration of the rifamycin sensitive disorder, such as but not limited to bowel or gastrointestinal disorder or changing how a patient responds to the rifamycin sensitive disorder, such as but not limited to bowel or gastrointestinal disorder.

As used herein, and unless otherwise specified, a “therapeutically effective amount” of a compound is an amount sufficient to provide any therapeutic benefit in the treatment or management rifamycin sensitive disorders, such as but not limited to bowel or gastrointestinal disorders or to delay or minimize one or more symptoms associated with rifamycin sensitive disorders, such as but not limited to bowel or gastrointestinal disorders. A therapeutically effective amount of a compound means an amount of the compound, alone or in combination with one or more other therapies and/or therapeutic agents that provide any therapeutic benefit in the treatment or management of rifamycin sensitive disorders, such as but not limited to bowel or gastrointestinal disorders, diarrhea or related diseases or disorders. The term “therapeutically effective amount” can encompass an amount that alleviates rifamycin sensitive disorders, such as but not limited to bowel or gastrointestinal disorders, improves or reduces rifamycin sensitive disorders, such as but not limited to bowel or gastrointestinal disorders, improves overall therapy, or enhances the therapeutic efficacy of another therapeutic agent. By way of example but not limitation, in one embodiment, the therapeutic benefit is inhibiting a bacterial infection or prolonging the survival of a subject with such a bacterial infection beyond that expected in the absence of such treatment.

As used herein, and unless otherwise specified, a “prophylactically effective amount” of a compound is an amount sufficient to prevent or delay the onset of rifamycin sensitive disorders, such as but not limited to bowel or gastrointestinal disorders, or one or more symptoms associated with rifamycin sensitive disorders, such as but not limited to bowel or gastrointestinal disorders or prevent or delay its recurrence. A prophylactically effective amount of a compound means an amount of the compound, alone or in combination with one or more other treatment and/or prophylactic agent that provides a prophylactic benefit in the prevention of a rifamycin sensitive disorder, such as but not limited to bowel or gastrointestinal disorders. The term “prophylactically effective amount” can encompass an amount that prevents rifamycin sensitive disorder, such as but not limited to bowel or gastrointestinal disorder or a related disease or disorder, improves overall prophylaxis, or enhances the prophylactic efficacy of another prophylactic agent. The “prophylactically effective amount” can be prescribed prior to, for example, travel to a location in which gastrointestinal disorders or diarrhea are common.

As used herein, “patient” or “subject” includes organisms which are capable of suffering from a rifamycin sensitive disorder, such as but not limited to bowel or gastrointestinal disorders or other disorder treatable by rifaximin or other poorly soluble members of the rifamycin group of antibiotics, such as rifabutin, rifapentine or rifampicin derivatives or who could otherwise benefit from the administration of a rifaximin as described herein, such as human and non-human animals. Preferred human animals include human subjects. The term “non-human animals” as used in the present disclosure includes all vertebrates, e.g., mammals, e.g., rodents, e.g., mice, and non-mammals, such as non-human primates, companion animals and livestock, e.g., sheep, dog, cow, chickens, amphibians, reptiles, etc. Susceptible to a rifamycin sensitive disorder is meant to include, but not be limited to, subjects at risk of developing a bowel or gastrointestinal disorder or infection, e.g., subjects suffering from one or more of an immune suppression, subjects that have been exposed to other subjects with a bacterial infection, physicians, nurses, subjects traveling to remote areas known to harbor bacteria that causes travelers' diarrhea, subjects who drink amounts of alcohol that damage the liver, subjects with a history of hepatic dysfunction, etc.

The term “polymorphism,” as used herein, refers to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compactability and/or x-ray diffraction peaks. The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see Wall, G. M. Pharm Manuf 3, 33, 1986); Haleblian, J. K. and McCrone, W. J. Pharm. Sci., 58, 911 (1969); and Haleblian, J. K., J. Pharm. Sci., 64, 1269, 1975). As used herein, the term polymorph is occasionally used as a general term in reference to the forms of rifaximin and includes within the context, salt, hydrate, polymorph co-crystal and amorphous forms of rifaximin. This use depends on context and will be clear to one of skill in the art. Exemplary rifaximin polymorphs include, for example, forms alpha, beta, gamma, delta, epsilon, iota, zeta, eta and amorphous. Also, terms such as “element” or “component” encompass both elements and components comprising one unit and elements and components that comprise more than one subunit unless specifically stated otherwise. Also, the use of the term “portion” can include part of a moiety or the entire moiety.

The rifamycin class of antibiotics are well known and include but are not limited to rifamycin A, B, C, D, E, S and SV and derivatives thereof, some of which, for example, rifampicin (rifampin), rifabutin, rifapentine and rifaximin are currently marketed antibiotic therapeutics (see for example the following patent documents: U.S. Pat. Nos. 3,625,960; 3,625,961; 3,817,986; 3,865,812; 3,884,763; 3,901,764; 3,923,791; 3,933,800; 3,933,801; 3,963,705; 4,002,752; 4,005,076; 4,005,077; 4,042,683; 4,108,853; 4,124,585; 4,124,586; 4,150,023; 4,169,834; 4,188,321; 4,193,920; 4,217,277; 4,217,278; 4,261,891; 4,312,866; 4,353,826; 4,431,735; 4,447,432; 4,507,295; 4,590,185; 4,880,789; 6,476,036; 7,229,996; 7,247,634; 7,678,791; 7,709,634; U.S. Patent Publication Nos.: US20060019985; US20080139577; US20090082558; US20090324736; US20100204173; WIPO Patent Publication Nos.: WO/2000/025721; WO/2005/020894; WO/2007/103448; WO/2008/008480; WO/2008/016708; WO/2008/035109; WO/2010/044093; WO/2009/008005; WO/2010/067072; WO/2008/048298; WO/2009/001060; WO/2009/010763; WO/2009/108730; WO/2009/108814; WO/2009/137672; WO/2010/040020; WO/2010/005836; WO/2010/122436 and EP0228606, all of which are hereby incorporated herein by reference). Rifamycins are used alone or in combination with other agents, to treat or prevent infections with, Gram-positive and some Gram-negative bacteria. They are used, alone or in combination with other agents, to treat or prevent infections with, but not limited to, N. meningitides, H. influenzae Type b, Chlamydophila pneumonia, Staph. aureus, Strep. epidermidis as well as diseases caused by highly resistant Mycobacteria, such as Mycobacterium tuberculosis, M. leprae, M. avium intracellulare, M. kansasii, and M. marinum.

The following discussion is directed to various embodiments of the present disclosure. Although one or more of these embodiments may be preferred for some applications, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Methods and compositions described herein are useful for increasing the solubility and activity of antibiotics of the rifamycin class (e.g., a pyrido-imidazo rifamycin), which includes rifabutin, rifapentine, rifampicin (rifampin), rifaximin and derivatives thereof, in particular those that are less soluble in an aqueous environment, such as rifaximin. The disclosed methods and compositions are based on the inventors' discovery that the addition of bile acids resulted in an increase in the solubility of rifaximin, a poorly soluble rifamycin antibiotic and that solubility increased with increasing concentrations of bile acid. Bile acids contain structural components that are hydrophilic on one side and hydrophobic at the other. The amphipathic nature of bile acids enables them to self-associate in water to form polymolecular aggregates. Each micelle contains 4-50 molecules depending on the type and structure, which can solubilize other lipids as well as hydrophobic molecules in the form of mixed micelles. Properties of bile acids are described by Cohen et al., 1990 (Cohen, D. E., et al., Structural alterations in lecithin-cholesterol vesicles following interactions with monomeric and micellar bile salts: physical-chemical basis for subselection of biliary lecithin species and aggregative states of biliary lipids during bile formation. J Lipid Res 31: 55-70, 1990).

Rifaximin (INN; see The Merck Index, XIII Ed., 8304) is a largely insoluble antibiotic belonging to the rifamycin class of antibiotics, e.g., a pyrido-imidazo rifamycin that has broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J., et al. Pharmacokinetic study of rifaximin after oral administration in healthy volunteers. Int J Clin Pharmacol Res, 14 (2), 51-56, 1994).

Rifaximin is described in, among other places, Italian Patent IT 1154655 and EP 0161534. EP 0161534 discloses a process for rifaximin production using rifamycin 0 as the starting material (The Merck Index, XIII Ed., 8301). U.S. Pat. No. 7,045,620 and PCT Publication WO 2006/094662 disclose polymorphic forms of rifaximin. Rifaximin is currently available in the U.S. under the brand name XIFAXAN by Salix Pharmaceuticals. It is also sold in Europe under the names SPIRAXIN, ZAXINE, NORMIX, RIFACOL and COLIDUR and in India under the name RIXMIN. However, it is expensive and many in the world suffering life threatening bowel or gastrointestinal disorders cannot afford therapy. Therefore, there is a longstanding need to reduce the cost associated with rifaximin therapy. Furthermore, rifaximin has been shown to have minimal effect on bacterial flora or the infecting bacteria in the aqueous environment of the colon. Therefore, new methods and formulations that increase the efficacy of rifaximin are necessary for the treatment of certain infections, such as, for example, infection in the colon. The ability to expand the activity of rifaximin, such that it can cover a greater potion or the entire intestinal track will increase its utility in treating disorders such as, but not limited to, irritable bowel syndrome and Crohn's disease.

Rifaximin is a compound having the structure of formula I: (I).

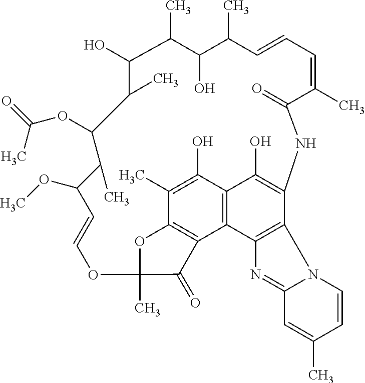

Rifaximin can be used to treat many bowel or gastrointestinal related disorders including, but not limited to, one or more of irritable bowel syndrome, diarrhea, microbe associated diarrhea, Clostridium difficile associated diarrhea, travelers' diarrhea, small intestinal bacterial overgrowth, Crohn's disease, diverticular disease, chronic pancreatitis, pancreatic insufficiency, enteritis, colitis, hepatic encephalopathy, minimal hepatic encephalopathy or pouchitis. Topical skin infections and vaginal infections may also be treated with the rifaximin forms described herein. In a specific embodiment, the instant methods provide for treating infections of the colon using rifaximin and one or more bile acids. The length of treatment for a particular bowel disorder will depend in part on the disorder. For example, travelers' diarrhea may only require treatment duration of 12 to about 72 hours, while Crohn's disease may require treatment durations from about 2 days to 3 months.

Because the insolubility of rifaximin is well known, rifaximin was chosen to exemplify the ability of bile salt addition to enhance the activity of poorly soluble rifamycin derivatives.

Specifically, the bile salt addition to enhance the activity of rifaximin on Enterotoxogenic E. coli, Enteroaggregative E. coli, Shigella flexneri and Salmonella enteric. Without wishing to be bound by any particular scientific theory, it is thought that rifaximin acts by binding to the beta-subunit of the bacterial deoxyribonucleic acid-dependent ribonucleic acid (RNA) polymerase, resulting in inhibition of bacterial RNA synthesis. It is active against numerous gram positive and gram negative bacteria, both aerobic and anaerobic. In vitro data indicate rifaximin is active against species of Staphylococcus, Streptococcus, Enterococcus, and Enterobacteriaceae. Bacterial reduction or an increase in antimicrobial resistance in the colonic flora does not frequently occur following treatment with rifaximin and does not have a clinical importance.

Cholanology, the study of bile acids, and particularly bile acid chemistry has been of interest for the better part of a century. Although much is known, bile acid chemistry involves a wide variety of chemical entities, many with surprising properties (for a review see, for example, Mukhopadhyay and Maitra, Chemistry and biology of bile acids, Current Science 87: 1666-1683, 2004) Pharmaceutical grade bile acid preparations are commercially available at relatively low cost. This low cost is due to the fact that the bile acids are obtained from animal carcasses, particularly large animals such as cows and sheep.

Bile acids are biosynthesized in the liver from cholesterol through a multi-step enzymatic process and form a major part of the organic component of bile. Bile acids are highly hydrophobic with a perhydrocyclopentanophenanthrene steroid nucleus consisting of three six-membered rings fused to a fourth five-membered ring. Following secretion, the primary bile acids (chenodeoxycholic and cholic acids) undergo conjugation through a peptide linkage with either taurine (tauroconjugation) or glycine (glycoconjugation). The ratio of glycoconjugates to tauroconjugates in human bile can be as high as 9:1 in rural African women and as low as 0.1:1 in tauro-fed subjects. The conjugated bile acids further undergo modification by the indigenous lumenal bacterial flora during their intestinal transit mainly through deconjugation, 7α-dehydrogenation (chenodeoxycholic acid to 7-oxolithocholic acid), and 7α-dehydroxylation (cholic acid to deoxycholic acid and chenodeoxycholic acid to lithocholic acid). Cholic, chenodeoxycholic, deoxycholic, lithocholic, glycocholic, and taurocholic acids are the most abundant bile acids found in human. The total bile acids concentration in the small bowel ranges from 2 mM to 30 mM depending on the diet and other metabolic conditions. Only 2-5% of the bile acids secreted in a normal human enter the colon after reabsorption in the ileum. Accordingly, in some embodiments, bile acids for use in disclosed compositions include, but are not limited to, cholic acid, chenodeoxycholic acid, deoxycholic acid, lithocholic acid, glycocholic acid, taurocholic acid and glyco-conjugates of the bile acids, such as glycocholic acid, glycodeoxycholic acid, glycochenodeoxycholic acid, taurocholic acid and taurodeoxycholic acid, glycolithocholic acid, taurolithocholic acid, taurohyodeoxycholic acid, and taurochenodeoxycholic acid as well as ursocholic acid, tauroursodeoxycholic acid, and glycoursodeoxycholic acid.

Provided herein are methods of treating, preventing, or alleviating rifamycin sensitive disorders, such as but not limited to bowel or gastrointestinal disorders or related disorders, or symptoms thereof, comprising administering to a subject in need thereof an effective amount of one or more of the solid dispersion forms. Bowel or gastrointestinal related disorders include, but are not limited to, one or more of irritable bowel syndrome, diarrhea, microbe associated diarrhea, Clostridium difficile associated diarrhea, travelers' diarrhea, small intestinal bacterial overgrowth, Crohn's disease, diverticular disease, chronic pancreatitis, pancreatic insufficiency, enteritis, colitis, hepatic encephalopathy, minimal hepatic encephalopathy or pouchitis. Topical skin infections and vaginal infections may also be treated with the rifaximin forms described herein. In a specific embodiment, are methods for treating infections of the colon using rifaximin and one or more bile acids. The length of treatment for a particular bowel disorder will depend in part on the disorder. For example, travelers' diarrhea may only require treatment duration of 12 to about 72 hours, while Crohn's disease may require treatment durations from about 2 days to 3 months. Dosages of rifaximin will also vary depending on the disease state. Proper dosage ranges are provided herein.

The identification of those subjects who are in need of prophylactic treatment for a bowel disorder is well within the ability and knowledge of one skilled in the art. Certain of the methods for identification of subjects which are at risk of developing a bowel disorder which can be treated by the subject method are appreciated in the medical arts, such as family history, travel history and expected travel plans, the presence of risk factors associated with the development of that disease state in the subject. A clinician skilled in the art can readily identify such candidate subjects, by the use of, for example, clinical tests, physical examination and medical, family or travel history.

Rifaximin was chosen as a representative example of a poorly soluble rifamycin derivative, and it is known in the art that rifaximin is not effective for treating colonic infection because of its known poor solubility in water. The data presented herein demonstrate that aqueous insolubility explains the lack of drug effect with rifaximin in the colon. Rifaximin's colonic bioavailability appears to be below the average MICs of most coliform flora (about 32 μg/mL) with the exception of colonic pathogens with lower MICs such as Clostridium difficile (MICS=0.025 μg/mL). However, as demonstrated herein, solutions comprising bile acids, alone or in combination, effectively solubilize rifaximin, thereby allowing for improved activity and therefore effectiveness as a treatment of colonic infections.

Accordingly, in a specific embodiment, a method of treating a subject having a colonic infection comprising administering to a subject in need thereof a therapeutically effective amount of a poorly soluble rifamycin, such as rifaximin, and one or more bile acids, to thereby treat the subject. Advantages of such an approach include, but are not limited to, efficacy in the colon, decreases in the required dose thus increasing the therapeutic effect by decreasing the likelihood of any off target toxicity. Such off target effects include, but are not limited to, hepatotoxicity and the induction of hepatic cytochrome P450 enzymes (such as CYP2C9 and CYP3A4) which effects the rate of metabolism of other drugs that are cleared by the liver, as is seen with the use of rifampicin. In accordance with certain embodiments, pharmaceutical compositions are provided comprising an effective amount of the rifamycin, such as but not limited to rifaximin, and one or more bile acids, and a pharmaceutically acceptable carrier.

Increased solubility offers the opportunity to decrease the amount of the rifamycin derivative applied. In a further embodiment, the effective amount is effective to treat a bacterial infection or a bowel or gastrointestinal related disorder such as, but not limited to, small intestinal bacterial overgrowth, Crohn's disease, hepatic encephalopathy, antibiotic associated colitis, and/or diverticular disease, irritable bowel syndrome, diarrhea, microbe associated diarrhea, Clostridium difficile associated diarrhea, travelers' diarrhea, diverticular disease, chronic pancreatitis, pancreatic insufficiency, enteritis, colitis, minimal hepatic encephalopathy or pouchitis (see for example, among others US20100048520, EP2257557, WO/2010/040020 and WO/2009/108814) and for treating hepatic encephalopathy with rifaximin, see, for example, N. Engl J Med.: 362: 1071-1081, 2010 (see for example, U.S. patent publication US20100204173).

Certain embodiments also provide pharmaceutical compositions comprising rifamycins, such as rifaximin, one or more bile acids, and a pharmaceutically acceptable carrier. In some cases the pharmaceutical composition further comprises an excipient such as, but not limited to, one or more of a diluting agent, binding agent, lubricating agent, disintegrating agent, coloring agent, flavoring agent or sweetening agent. One composition may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In some embodiments, rifamycins, such as rifaximin, are administered to the subject using a pharmaceutically-acceptable formulation, e.g., a pharmaceutically-acceptable formulation comprising one or more bile acids that provides sustained delivery of the rifamycin, such as rifaximin, polymorph to a subject for at least, 2 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for oral administration to a subject. In other embodiments, as described in detail below, the pharmaceutical compositions disclosed may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a suitable sterile solution or suspension; (3) topical application, for example, as a suitable cream, ointment or spray applied to the skin; (4) intravaginally or intrarectally, for example, as a suitable pessary, suppository, cream or foam; or (5) aerosol, for example, as suitable aqueous aerosol, liposomal preparation or solid particles containing the compound.

The phrase “pharmaceutically acceptable” refers to those rifamycins, such as rifaximin, and one or more bile acids of the presently disclosed methods, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is preferably “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a rifamycin, such as rifaximin forms disclosed herein, include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred %, this amount will range from about 1% to about ninety-nine % of active ingredient, preferably from about 5% to about 80%, or from about 10% to about 60%.

Methods of preparing these compositions include the step of bringing into association the rifamycin, such as rifaximin, and bile acids with the carrier and, optionally, one or more accessory ingredients.

Compositions suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of rifamycin, such as rifaximin, and one or more bile acids. A compound may also be administered as a bolus, electuary or paste.

Rifamycin, such as rifaximin, and one or more bile acids as disclosed herein can be advantageously used in the production of medicinal preparations having antibiotic activity. Medicinal preparations for oral use will contain rifamycin, such as rifaximin, together with one or more bile acids and the usual excipients, for example diluting agents such as mannitol, lactose and sorbitol; binding agents such as starches, gelatins, sugars, cellulose derivatives, natural gums and polyvinylpyrrolidone; lubricating agents such as talc, stearates, hydrogenated vegetable oils, polyethylenglycol and colloidal silicon dioxide; disintegrating agents such as starches, celluloses, alginates, gums and reticulated polymers; coloring, flavoring and sweetening agents.

Some embodiments of the disclosed compositions include solid preparations administrable by the oral route, for instance coated and uncoated tablets, of soft and hard gelatin capsules, sugar-coated pills, lozenges, wafer sheets, pellets and powders in sealed packets or other containers.

Medicinal preparations for topical use can contain rifamycin, such as rifaximin, together with one or more bile acids and the usual excipients, such as white petrolatum, white wax, lanoline and derivatives thereof, stearylic alcohol, propylene glycol, sodium lauryl sulfate, ethers of fatty polyoxyethylene alcohols, esters of fatty polyoxyethylene acids, sorbitan monostearate, glyceryl monostearate, propylene glycol monostearate, polyethylene glycols, methylcellulose, hydroxymethyl propylcellulose, sodium carboxymethylcellulose, colloidal aluminium and magnesium silicate, sodium alginate. Thus certain embodiments relate to all types of the topical preparations, for instance ointments, pomades, creams, gels and lotions.

In solid dosage forms of rifamycin, such as rifaximin, for oral administration (capsules, tablets, pills, dragees, powders, granules and the like), the active ingredient is typically mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of rifamycin, such as rifaximin, and one or more bile acids include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the oral compositions can include adjuvants such as wetting agents, emulsifying and suspending agents, sweetening, flavoring, coloring, perfuming and preservative agents.

Pharmaceutical compositions for rectal or vaginal administration may be presented as a suppository, which may be prepared by mixing rifamycin, such as rifaximin, and one or more bile acids with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Compositions which are suitable for vaginal administration also include pessaries, tampons, creams, gels, pastes, foams or spray formulations containing such carriers as are known in the art to be appropriate (many such formulations are known, such as those described in U.S. Pat. Nos. 4,384,003; 5,246,704; 5,409,710; 6,139,863; 6,491,942; 7,749,488, among others).

Dosage forms for the topical or transdermal administration of rifamycin, such as rifaximin, include powders, sprays, ointments, pastes, creams, lotions, gels, solutions, patches and inhalants. The rifamycin, such as rifaximin, and one or more bile acids may be mixed under sterile conditions with a pharmaceutically-acceptable carrier, and with any preservatives, buffers, or propellants which may be required.

Ointments, pastes, creams and gels may contain, in addition to rifamycin, such as rifaximin, and one or more bile acids, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to rifamycin, such as rifaximin, and one or more bile acids, excipients such as lactose, talc, silicic acid, aluminium hydroxide, calcium silicates and polyamide powder, or mixtures of these substances. Sprays can additionally contain customary propellants, such as chlorofluorohydrocarbons and volatile unsubstituted hydrocarbons, such as butane and propane.

The rifamycin (for example, rifaximin) and one or more bile acids can be alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

An aqueous aerosol is made, for example, by formulating an aqueous solution or suspension of the agent together with conventional pharmaceutically-acceptable carriers and stabilizers. The carriers and stabilizers vary with the requirements of the particular compound, but typically include non-ionic surfactants (Tweens, Pluronics®, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of rifamycin, such as rifaximin, and one or more bile acids to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the disclosure

Pharmaceutical compositions suitable for parenteral administration may comprise rifamycin, such as rifaximin, and one or more bile acids in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, to prolong the effect of a drug, it is desirable to alter the absorption of the drug. This may be accomplished by the use of a liquid suspension of crystalline, salt or amorphous material having poor water solubility. The rate of absorption of the drug may then depend on its rate of dissolution which, in turn, may depend on crystal size and crystalline form. Alternatively, delayed absorption of a drug form is accomplished by dissolving or suspending the drug in an oil vehicle.

Injectable depot forms are made by forming microencapsule matrices of rifamycin, such as rifaximin, to rifaximin and one or more bile acids in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the rifamycin (for example, rifaximin) and one or more bile acids are administered as pharmaceuticals, to humans and animals, they can be given per se or as a pharmaceutical composition containing, for example, 0.1 to 99.5% (or in some cases from 0.5 to 90%) of active ingredient in combination with a pharmaceutically-acceptable carrier.

Regardless of the route of administration selected, the rifamycin (for example, rifaximin) and one or more bile acids, which may be used in a suitable hydrated form, and/or the pharmaceutical compositions presently disclosed, are formulated into pharmaceutically-acceptable dosage forms by methods known to those of skill in the art.

Actual dosage levels and time course of administration of the active ingredients in the pharmaceutical compositions may be varied so as to obtain an amount of the active ingredient which is effective to achieve the desired therapeutic response for a particular subject, composition, and mode of administration, without being toxic to the subject. An exemplary dose range is from 25 to 3000 mg per day. Other doses include, for example, 600 mg/day, 1100 mg/day and 1650 mg/day. Other exemplary doses include, for example, 1000 mg/day, 1500 mg/day, from between 500 mg to about 1800 mg/day or any value in-between.

In some instances, a selected dose of the rifamycin (for example, rifaximin) and one or more bile acids disclosed herein is the maximum that a subject can tolerate without developing serious side effects. In many cases, the rifamycin (for example, rifaximin), and one or more bile acids of the presently disclosed methods, is administered at a concentration of about 1 mg to about 200 mg per kilogram of body weight, about 10 to about 100 mg/kg or about 40 mg to about 80 mg/kg of body weight. Ranges intermediate to the above-recited values are also intended to be part. For example, doses may range from 50 mg to about 2000 mg/day.

In combination therapy treatment, a disclosed rifamycin compound and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment in which another therapeutic agent is administered to an animal, the effective amount of the compound of this disclosure is less than its effective amount in case the other therapeutic agent is not administered. In another embodiment, the effective amount of the conventional agent is less than its effective amount in case the compound of this disclosure is not administered. In this way, undesired side effects associated with high doses of either agent may be minimized. Other potential advantages (including without limitation improved dosing regimens and/or reduced drug cost) will be apparent to those skilled in the art.

In various embodiments, the therapies (e.g., prophylactic or therapeutic agents such as but not limited to a rifamycin, such as rifaximin) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same subject's visit.

In certain embodiments, one or more compounds and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same compounds may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than the rifamycin, such as rifaximin, may be repeated and the administration may be separated by at least 1 day, 2 days, 3 days, 5 days, 10 days, 15 days, 30 days, 45 days, 2 months, 75 days, 3 months, or at least 6 months.

Certain indications may require longer treatment times. For example, travelers' diarrhea treatment may only last from between about 12 hours to about 72 hours, while a treatment for Crohn's disease may be from between about 1 day to about 3 months. A treatment for hepatic encephalopathy may be, for example, for the remainder of the subject's life span. A treatment for IBS may be intermittent for weeks or months at a time or for the remainder of the subject's life.

### EXAMPLES

### Example 1

### Bile Acids Improve the Effects of Rifaximin

Diarrhea is one of the most common illnesses of international travelers; occurring in 20-50% of persons visiting developing regions from, countries. The diarrhea-producing E. coli important in Travelers' diarrhea are enterotoxigenic Escherichia coli (ETEC) and enteroaggregative E. coli (EAEC); both ETEC and EAEC are known to be small bowel pathogens. ETEC is the most common causative agent identified in approximately half of the cases of travelers' diarrhea. It is also the most commonly isolated bacterial enteropathogen in children under 5 years in developing countries and responsible for approximately 200 million diarrheal episodes and 380,000 deaths annually. ETEC colonizes the intestinal lumen by binding to specific receptors on the enterocytic surfaces. It produces two notable enterotoxins that cause pathology; a cholera-like heat-labile toxin and small molecular weight heat-stable toxin. Therefore the ability to inhibit the growth of ETEC was considered an important parameter for success.

Rifaximin, a largely water-insoluble, non-absorbable (<0.4%), bacterial RNA synthesis inhibitory drug has been shown to be safe and effective for the treatment of Travelers' diarrhea caused by diarrhea-producing Escherichia coli. However, rifaximin has minimal effects on colonic flora, and this is likely related to the drug's insolubility in water due to its hydrophobic properties and the aqueous environment of the colon. The purpose of this analysis was to establish the antimicrobial effect and bioavailability of rifaximin in aqueous solutions in the presence and absence of physiologic concentrations of bile acids.

Enterotoxigenic Escherichia coli (ETEC) strain H10407 was used as a model bacteria in all the susceptibility experiments. This strain produces both the heat labile and heat stable enterotoxins (LT and ST) important in the pathogenesis of travelers' diarrhea. The in vitro minimal inhibitory concentration (MIC) of rifaximin to the test strain ranges between 32-128 μg/mL (DuPont, H. L., et al. Clin Microbiol Infect 10:1009-11, 2004; DuPont, H. L. et al. Ann Intern Med 142:805-12, 2005; Ruiza, J. et al., 59: 473-475, 2007, Jiang, Z. D. et al. (2005). Chemotherapy. 51 (suppl 1): 67-72; Sierra, J. M. et al. Antimicrob Agent and Chemoth. 45(2): 643-644; 2001 and Brook, I., J. Clin. Microbiol. 27: 2373-2375; 1989).

Rifaximin powder was obtained from Salix Pharmaceuticals (Morrisville, N.C.) and bile acids (cholic, chenodeoxycholic, deoxycholic, glycocholic, lithocholic, and taurocholic acids) were purchased from Sigma Aldrich (St. Louis, Mo.). Equimolar concentrations (0.67 mM) of each of these bile acids were pooled (4 mM total bile acids) for the experiments. In order to determine the Total Human Bile Concentration, a sample of human bile taken after cholecystectomy was kindly provided by Dr. David Graham (Department of Medicine, Veterans Affairs Medical Center and the Division of Molecular Virology, Baylor College of Medicine, Houston, Tex., and Director of Study Design and Clinical Research Core, Texas Medical Center Digestive Disease. Center). The total bile acids concentration was determined using the Diazyme Total Bile Acids assay (Diazyme Laboratories, CA) following the protocol provided by the manufacturer.

Solubility of Rifaximin: The solubility of rifaximin in different concentrations of synthetic bile acids and water, as solvents, was determined spectrophotometricly. In order to obtain a standard curve, rifaximin within a concentration range of 0.01 mg/mL to 20 mg/mL in 100% acetone was diluted serially with 100% ethanol. The sample was prepared by adding 12 mg of rifaximin powder to deionized water, 2.5 mM, 5 mM, 7.5 mM, 10 mM, 15 mM and 20 mM synthetic bile acids mixture (pH 7.4). The tubes were incubated for an hour and centrifuged at 16000×g for 30 minutes to eliminate undissolved particles. Absorbance measurements at 450 nm of both standards and samples were taken simultaneously using a Multiskan EX spectrophotometer (Thermo Scientific, Waltham, Mass.). A regression equation from the standard curve was used to estimate the concentration of rifaximin sample in deionized water and bile acids.

The percent solubility was calculated as:

$$[ \frac{Milligrams of rifaximin determined spectrophotometrically}{Milligrams of rifaximin dissolved} ] ( 100 \% )$$

Efficacy of Rifaximin Dose Response: Escherichia coli H10407 strain was grown in Luria Bertani (LB) media overnight to optical density at 600 nm (OD₆₀₀) of 0.5-1.0. Test groups consisted of a 30 mL LB media containing rifaximin, human bile or synthetic bile acids (Cholic, chenodeoxycholic, deoxycholic, glycocholic, lithocholic, and taurocholic acids) and E. coli H10407. Rifaximin powder was added to LB media containing synthetic bile acids or human bile at pH 7.4 and incubated for 30 minutes at ambient temperature on a magnetic stirrer. Using the Diazyme assay, the total bile acids concentration of human bile was determined to be approximately 39.7 mM. A tenth dilution (about 4 mM) of the human bile when added to rifaximin resulted in a significant increase in the antimicrobial effect of the drug. Based on this observation, 4 mM of total bile acids was used for all the experiments. In each experiment, an overnight culture of E. coli H10407 was added to the LB media to OD₆₀₀ of 0.04 (approximately 4×10⁷ cells/mL) and incubated for 4-6 hours at 37° C. in a shaker at 200 rpm. Optical density measurements (OD₆₀₀) were determined every 30 minutes. The experiment was replicated four times and average OD used for the analysis.

Beta-Galactosidase Assay: Beta-galactosidase is an essential enzyme required by E. coli to metabolize lactose during conditions when available glucose levels are low, but lactose levels are high. When expression of beta-galactosidase is inhibited in a media containing limiting amounts of glucose but high amounts of lactose, once the glucose in the media is exhausted bacterial death occurs. Rifaximin is believed to act by binding to the beta-subunit of bacterial DNA-dependent RNA polymerase resulting in inhibition of RNA synthesis and ultimately, protein synthesis. The degree of inhibition of RNA and protein synthesis depends upon the amount of rifaximin present, for example, within the culture media. Therefore, as an indirect measure of the bioavailability of rifaximin, E. coli H10407 were grown in the presence of rifaximin with and without bile acids under conditions in which the expression of beta-galactosidase would be induced. The ability of the rifaximin to inhibit the expression of beta-galactosidase was directly related to the ability of the added bile salts to increase the solubility and thus the activity of rifaximin. The beta-galactosidase assay was performed based on methodologies developed by Zhang and Bremer (Begley, M. et al., FEMS Microbiology Reviews. 29:625-651, 2005 and Miller (Steffen, R. et al., Am J. Gastroenterol. 98:1073-8, 2003) with some modifications. Briefly, the optical density (OD₆₀₀) of an overnight'culture was adjusted to 0.5. Aliquots (500 μL) of this culture were pipetted into different treatment tubes (in triplicate) containing 8 ug/mL, 16 μg/mL, and 32 μg/mL of rifaximin in 4 mM of total synthetic bile acids. The culture was induced to express β-galactosidase using 1 mM isopropyl β-D-1-thiogalactopyranoside (IPTG) and incubated on a shaker for 90 minutes at 30° C. The control group consisted of 500 μL culture, 1 mM IPTG, 8 ug/mL, 16 μg/mL, and 32 μg/mL rifaximin without bile acids. To, 1.5 mL microfuge tubes, 50 μL of the induced culture was added to 200 μL of permeabilization solution (0.8 mg/mL hexadecyl trimethylammonium bromide (CTAB), 0.4 mg/mL sodium deoxycholate, 200 mM dibasic sodium phosphate (Na₂HPO₄), 20 mM potassium chloride (KCl), 2 mM magnesium sulfate (MgSO₄), and 5.4 μL/mL beta-mercaptoethanol. After 30 minutes incubation at 30° C., 600 μL of substrate solution (60 mM Na₂HPO₄, 40 mM NaH₂PO₄, 10 mM KCl, 20 μg/mL CTAB, 1 mg/mL O-nitrophenyl-β-D-galactoside (ONPG), and 2.7 μL/mL β-mercaptoethanol) was added and the time of addition was recorded. The incubation was allowed to continue at 30° C. for 40 minutes, after which 350 μL of 2 M sodium carbonate (Na₂CO₃) was added to stop the reaction. The assay, tubes were centrifuged at 15000×g for 15 minutes and absorbance measurements at 420 nm were made in triplicate using the supernatants.

The concentration of β-galactosidase in Miller Units was calculated as:

$$[ \frac{( Absorbance at 420 nm of assay )}{( Absorbance at 600 nm of culture ) ( 0.05 mL ) ( Reaction time )} ] ( 1000 )$$

Effect of Bile Acids on Rifaximin Inhibition of E. coli H10407: The effect of rifaximin and bile acids on total protein expression by the bacteria during the first five hours of incubation was evaluated. An aliquot of this overnight culture (0.5 mL) with OD₆₀₀ of 0.5 was added either to 30 mL of LB media (pH 7.4) containing: (1) only rifaximin (16 μg/mL); (2) rifaximin and bile acids (16 μg/mL rifaximin in 4 mM total synthetic bile acids); (3) LB media only; or (4) 4 mM total synthetic bile acids. Aliquots (1 mL) of the culture in each tube were taken every hour for total protein determination. In order to lyse the cells and release the protein, 300 μL of permeabilization solution (2.4 mg/mL hexadecyl trimethylammonium bromide, 1.2 mg/mL sodium deoxycholate, 600 mM dibasic sodium phosphate, 60 mM potassium chloride, 6 mM magnesium sulfate, and 16 μL/mL beta-mercaptoethanol) was added. The tubes were incubated for one hour at room temperature and then centrifuged at 15000×g for 20 minutes. The protein concentration was determined in triplicate using the Bradford assay (Bradford, M. M., A rapid and sensitive method for the quantitation of microgram quantities of protein utilizing the principle of protein-dye binding. Anal Biochem. 72:248-54, 1976) using bovine serum albumin (BSA) as the standard. Each experiment was repeated three times with the average and standard deviation reported.

Statistical Analysis: To determine the significant level of the differences observed between the samples, Mann-Whitney two-tailed non-parametric tests of significance was performed using GraphPad Prism version 5.02 for Windows (GraphPad Software, San Diego, Calif.). In all cases, statistical significance was defined as p<0.05.

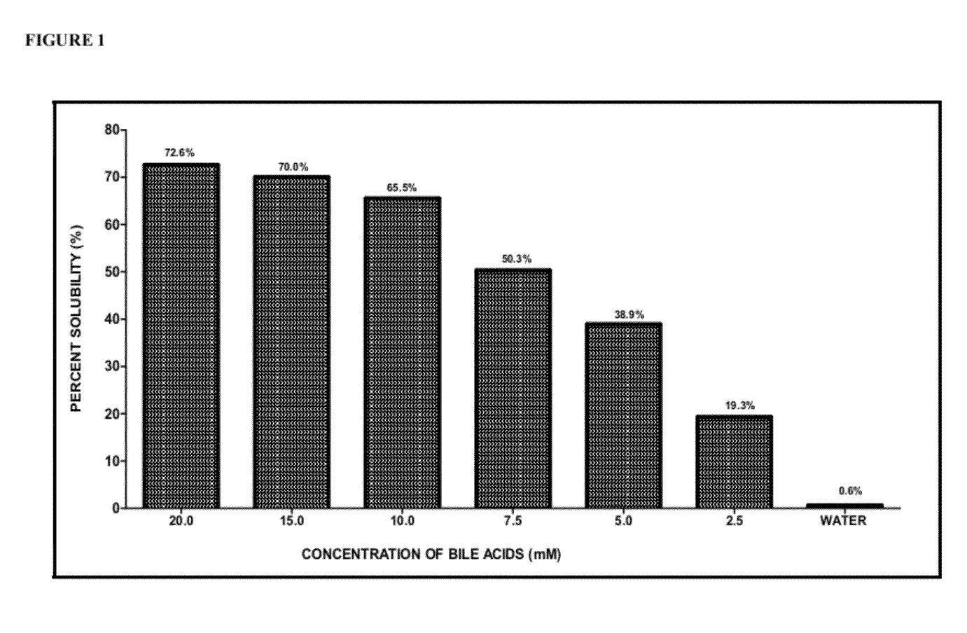

Solubility Improvement: To demonstrate the ability of bile acids to improve the solubility of rifaximin, the concentration of rifaximin that was in solution was determined spectrophotometricly at 450 nm. Different dilutions of rifaximin in 100% acetone were initially evaluated in the concentration range of 0.01 mg/mL to 20 mg/mL. In an aqueous solution, rifaximin was markedly more soluble when in the presence of bile acids. This is illustrated in FIG. 1, in which the solubility of 12 mg of rifaximin was shown to increase in water as the concentration of the bile acid mixture increased. The bile acid mixture was made up of equimolar concentrations of cholic, chenodeoxycholic, deoxycholic, sodium glycocholate, lithocholic, and taurocholic acids at pH 7.4. Thus it was determined that, within the concentration range analyzed, the aqueous solubility of rifaximin increased 70-120 fold when in the presence of bile acids.

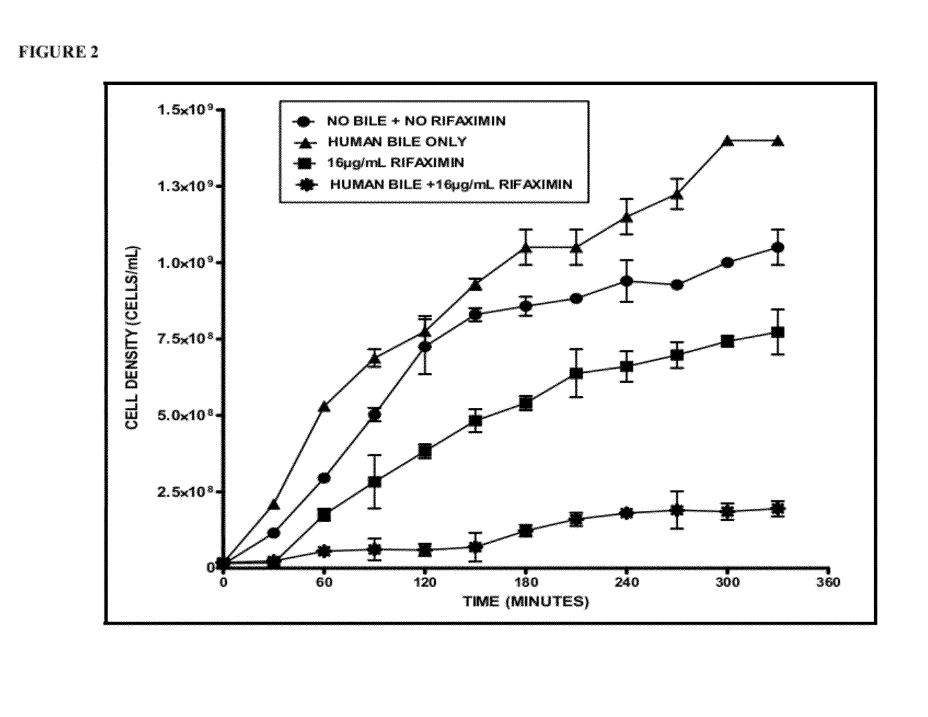

Activity Improvement: To confirm that the increased solubility of rifaximin in an aqueous environment due to the presence of bile acid translates into improved antimicrobial activity, the growth of E. coli H10407 was monitored in the presence of different concentrations of rifaximin at a constant total bile acids concentration that approximated physiologic levels. As an untreated control, ETEC Strain H10407 bacteria (without rifaximin or bile salts) were cultured LB media and a growth curve was determined by measuring the OD₆₀₀ at thirty minutes intervals during the incubation period. A second culture of ETEC Strain H10407 bacteria were cultured in LB media containing 16 μg/mL rifaximin. A third culture contained ETEC Strain H10407 bacteria in LB media, 16 μg/mL rifaximin and 30 mM of total bile acids (a mixture of 5 mM each of the bile acids: cholic, deoxycholic, chenodeoxycholic, glycocholic, lithocholic, and taurocholic acids at pH 7.4). A fourth culture contained ETEC Strain H10407 bacteria in LB media and the 30 mM of total bile acids only. The results, shown in FIG. 2, illustrate that at physiological pH and temperature, 30 mM of total bile acids had no inhibitory or killing effect on the bacteria cultures, in fact the presence of the bile acids tended to modestly enhance bacterial growth. The presence of rifaximin (16 μg/mL), even in the absence of bile acids, resulted in statistically significant (p=0.026, n=4) inhibition of bacterial growth during the incubation period. The effectiveness of rifaximin became more remarkable and improved significantly (p<0.0001, n=3) when bile acids were added.

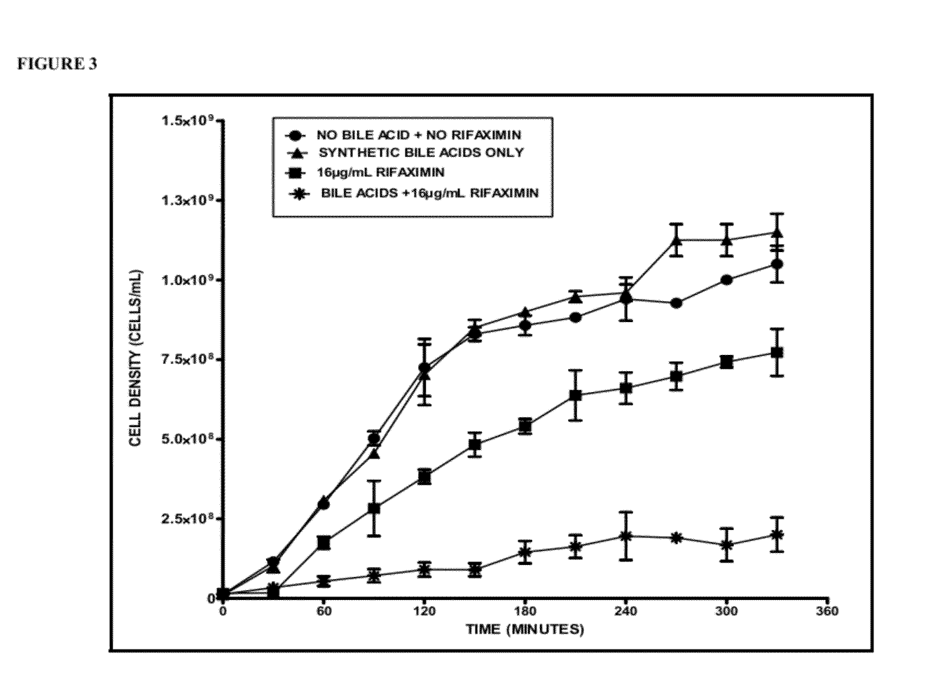

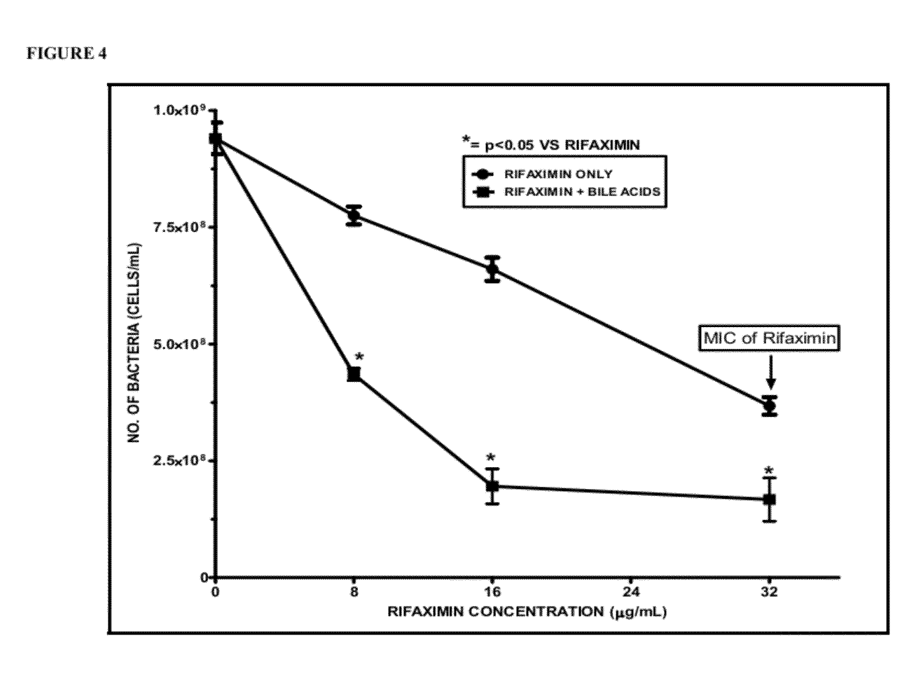

The inhibitory effect of rifaximin became more remarkable and improved significantly when bile acid (p=0.007 for synthetic bile acids and p=0.012 for human bile; n=4) was added. No significant difference (p=0.686, n=4) was observed between the effect of synthetic bile acids and human bile. It was determined that this bactericidal effect increased as the concentration of rifaximin (8 μg/mL-32 μg/mL) increased, even in the presence of constant bile acid concentrations (FIG. 3). After a 4 hour of incubation, cultures containing 8 μg/mL, 16 μg/mL, and 32 μg/mL of rifaximin in the presence of bile acids resulted in 3-fold, 4.5-fold, and 5.6-fold increase in bactericidal effect, respectively, as compared to cultures containing rifaximin but no bile acids. The increase in rifaximin activity observed in the presence of bile acids was even more notable at the lower concentrations of rifaximin tested. These findings illustrate that at physiologic temperature and pH, the addition of bile acids significantly increased the bactericidal effect of rifaximin. The antimicrobial effect increased with increasing concentration of rifaximin (8 μg/mL-32 μg/mL). Conversely, such a concentration curve was not feasible with increasing concentrations of bile acids due to their deleterious effect on the cells at higher concentrations (data not shown). After 4 hours of incubation, 8 μg/mL and 16 μg/mL of rifaximin containing synthetic bile acids resulted in 2-fold and 5.5-fold increase, respectively, in bacteriostatic effect over samples containing no bile acids (FIG. 4). The effect of bile acids was more notable at lower concentrations of rifaximin.

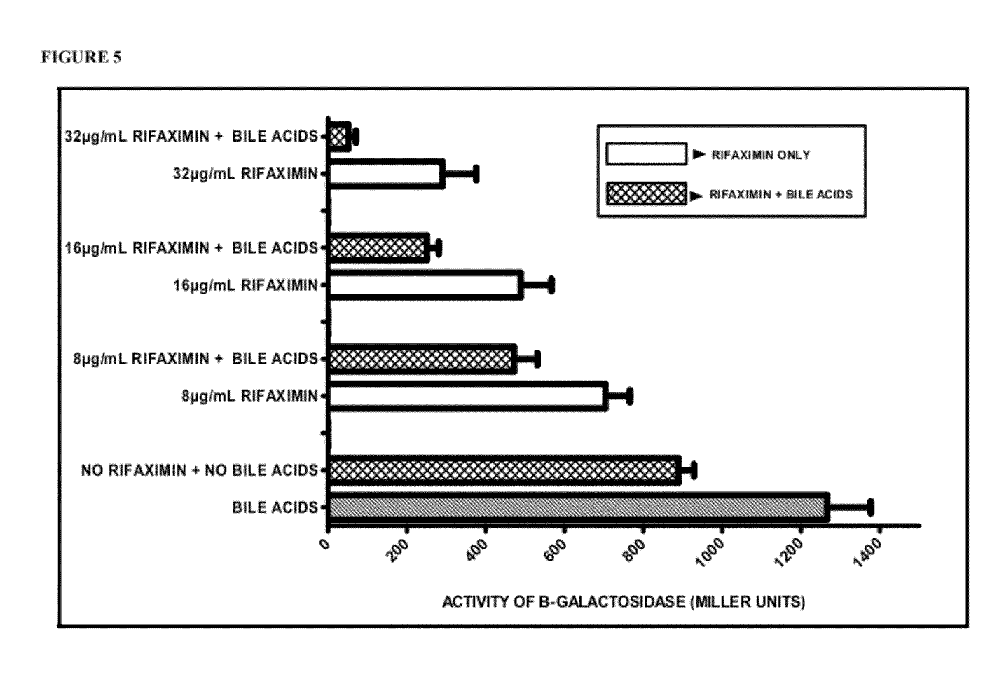

The degree of RNA synthesis inhibition in the test ETEC strain by rifaximin and bile acids was also determined. This was done by monitoring the level of beta-galactosidase expressed by E. coli H10407 grown in media containing rifaximin with and without bile acids. In the presence of bile acids and no rifaximin, expression of beta-galactosidase was higher (indicating a lack of inhibition) than the untreated cells (control), as shown in FIG. 5. On the other hand, the amount of beta-galactosidase produced decreased in a dose-dependent fashion below the control values when rifaximin was added to the culture medium. The beta-galactosidase level was, in turn, decreased below these already depressed values when the cells were treated with the combination of rifaximin and bile acids. The percent decrease in inhibition of beta-galactosidase expression in samples containing bile acids and rifaximin at concentration of 8 μg/mL, 16 μg/mL, and 32 μg/mL were 33%, 49%, and 82% respectively.

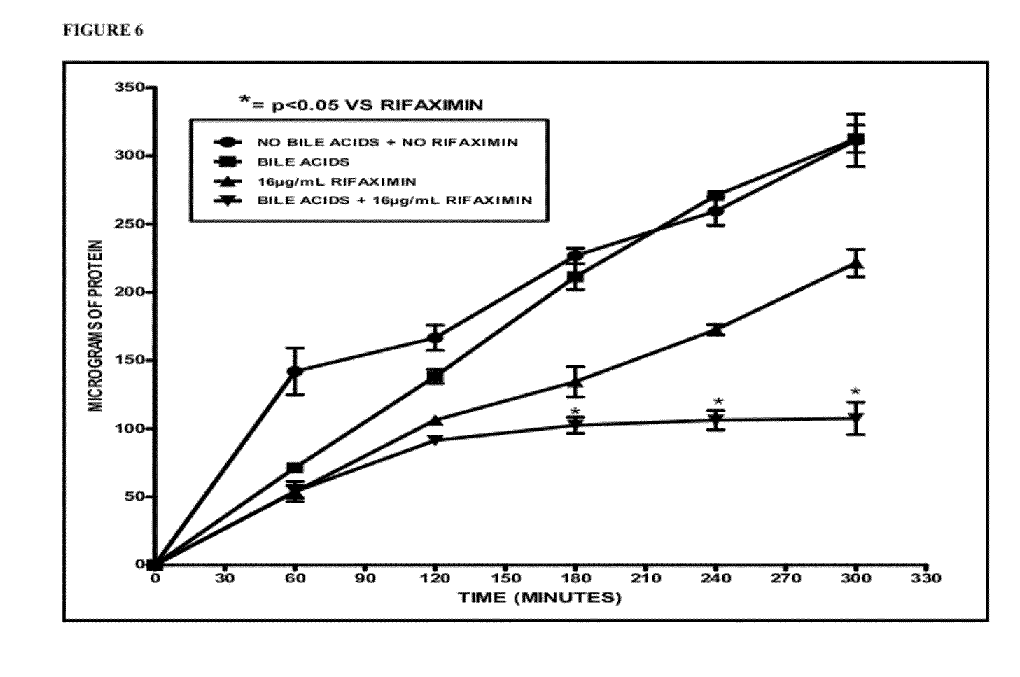

The total protein content of E. coli H10407 grown under different conditions of rifaximin and bile acids was monitored to further confirm the increased antimicrobial effect of rifaximin resulting from addition of bile acids (as observed from the other methods). After 4 hours of incubation, no significant difference (p=0.873, n=3) was observed in the amount of total bacterial protein expressed between samples treated with only bile acids and the control as shown in FIG. 6. Bacteria grown in a media containing both rifaximin and bile acids resulted in a lower amount of total protein than those without bile acids. The percent decrease in total bacterial protein expression after 4 hours incubation period when bile acids was added to media containing 16 μg/mL rifaximin was 59% compared to samples that contained equivalent amount of rifaximin without bile acids. This indicates that addition of bile acids to rifaximin makes the non-absorbable antibiotic more bioavailable to inhibit synthesis of an essential enzyme and proteins required for bacterial growth and that this inhibition occurred in a dose-response manner at previously sub-lethal concentrations of rifaximin.

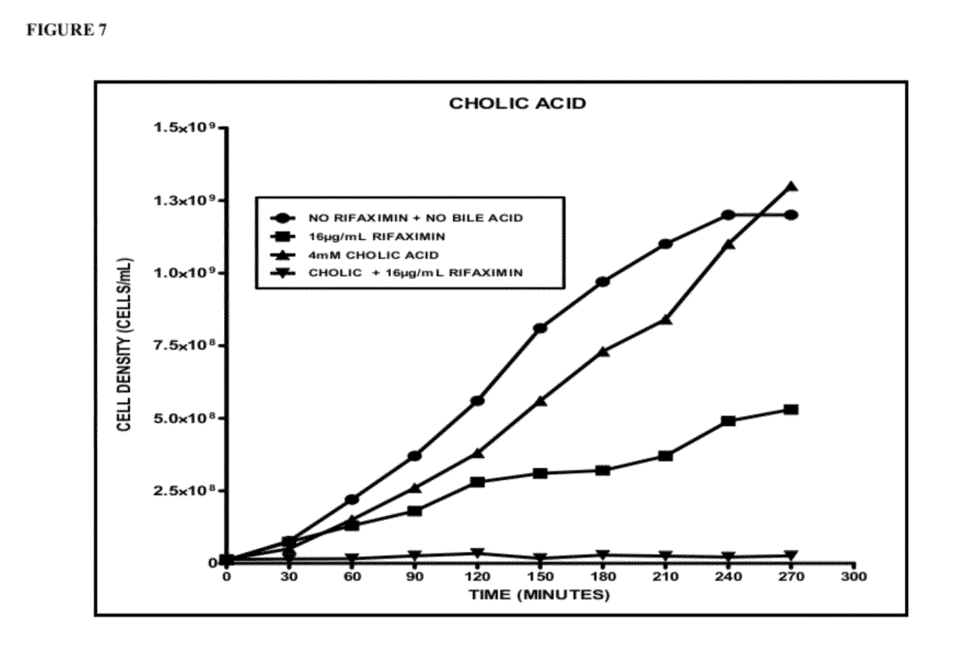

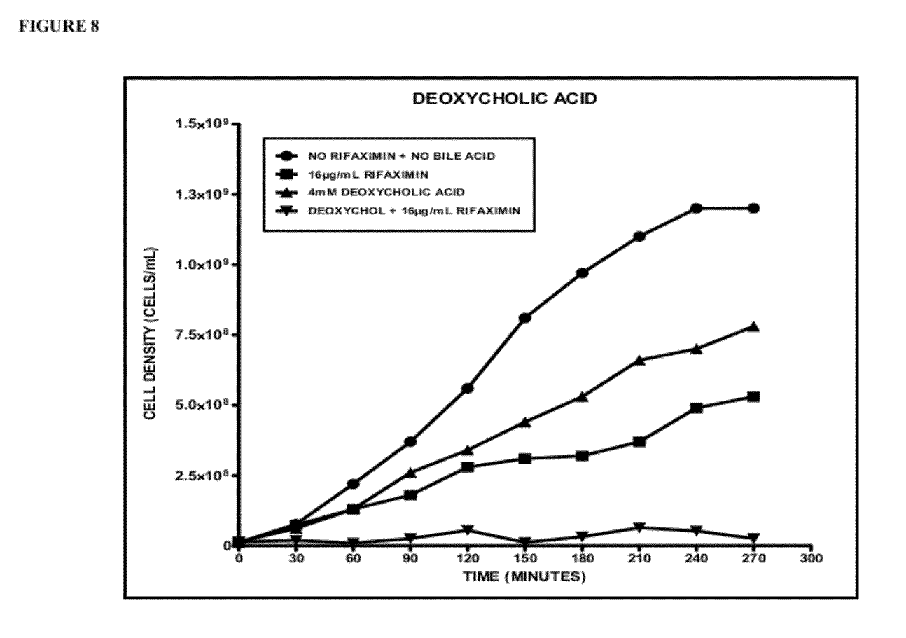

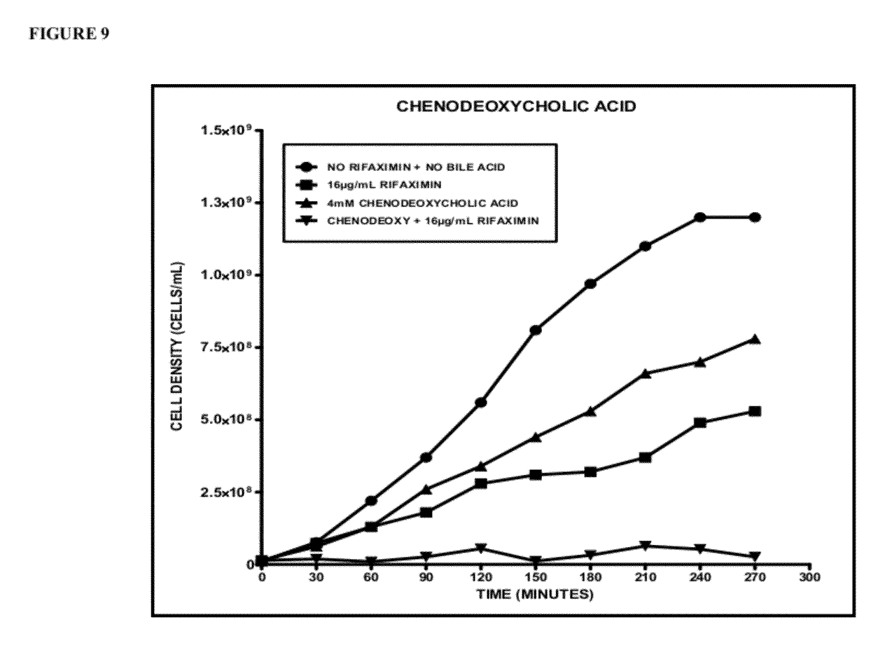

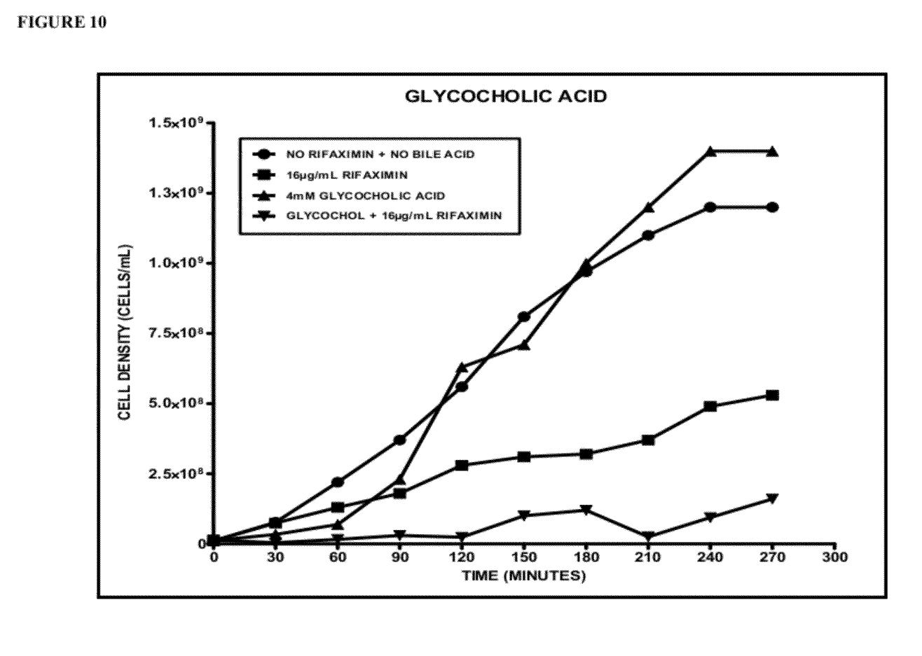

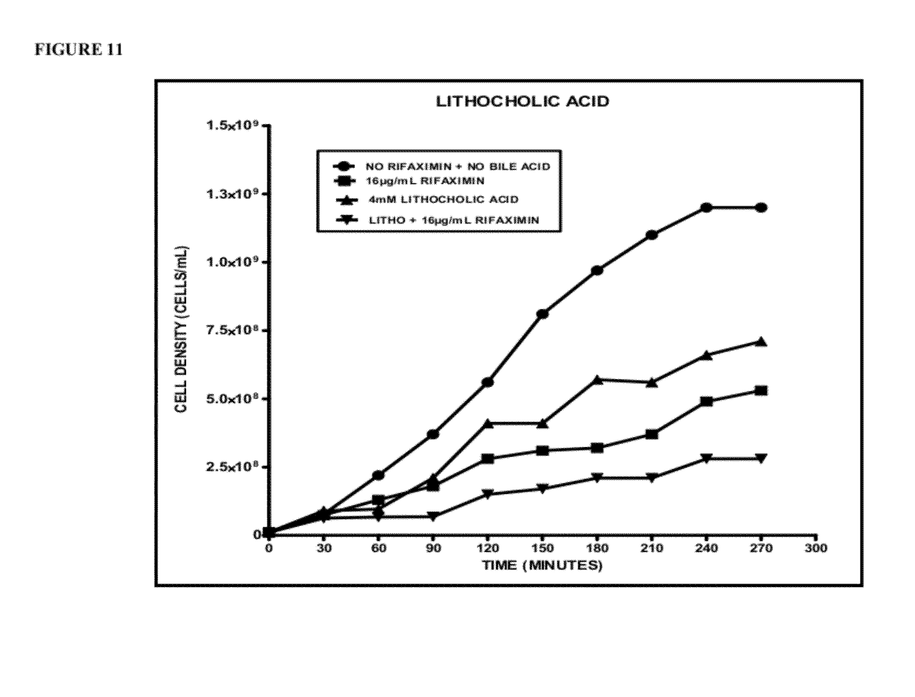

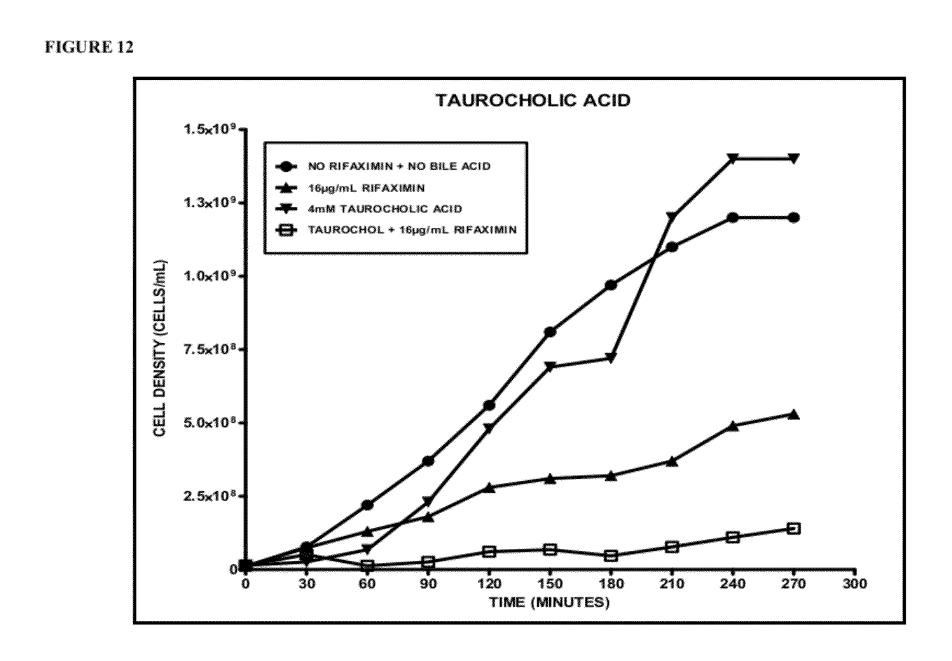

To appraise the potential physiological limitations due to the pooled equimolar concentrations of synthetic bile acids used, single bile acids (cholic, chenodeoxycholic, deoxycholic, glycocholic, lithocholic, and taurocholic acids) were evaluated (FIGS. 7-12). The E. coli H10407 cells were exposed to each of these bile acids in the presence and absence of sub-inhibitory concentration of rifaximin (16 μg/mL). The antimicrobial effect of rifaximin did not improve to a significant level on addition of 1 mM of each single bile acid (data not shown). However, 4 mM of each single bile acid increased the antimicrobial effect of rifaximin beyond that observed from both pooled synthetic bile acids and human bile (Table 1) except lithocholic acid. In each case, the cell density underwent further decrease in the presence of bile acid compared to cultures that contained only rifaximin. The percent decrease in cell density due to addition of cholic, deoxycholic, chenodeoxycholic, glycocholic, taurocholic, and lithocholic acids was 95.5%, 93.8%, 89.2%, 80.8%, 77.6%, and 43.9% respectively. Putting together, the data illustrates that at physiologic temperature and pH, bile acids significantly increased the antimicrobial effect of rifaximin.

### Example 2

### Increased Activity of Rifaximin on Other Bacterial Pathogens

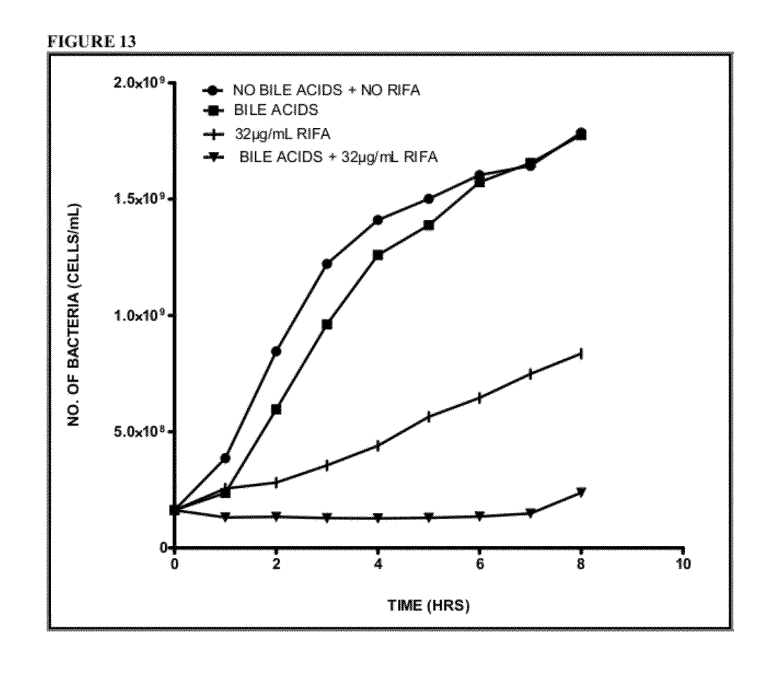

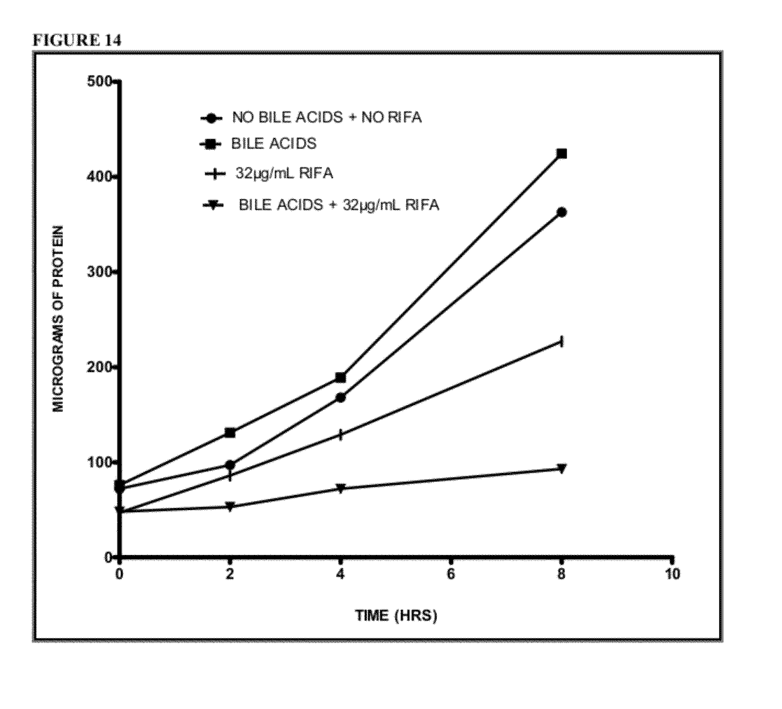

Shigella flexneri: The minimal inhibitory concentration (MIC) for the bacterial strain Shigella flexneri (serotype 2B) for rifaximin is 64 μg/mL. Therefore, a sub-lethal concentration of rifaximin (32 μg/mL) was used to establish that the addition of bile acids also enhanced the activity of rifaximin to inhibit the growth of Shigella flexneri (serotype 2B) as shown in FIG. 13 and protein synthesis as shown in FIG. 14.

The untreated control contained Shigella flexneri (serotype 2B) (without rifaximin or bile acids) cultured in LB media and a growth curve was determined by measuring the OD₆₀₀ at sixty minutes intervals during the incubation period (as described previously). A second culture of Shigella flexneri (serotype 2B) bacteria in LB media to which was added 4 mM of bile acids (comprising an equimolar mixture of each of the bile acids: cholic, deoxycholic, chenodeoxycholic, glycocholic, lithocholic, and taurocholic acids at pH 7.4). A third culture contained Shigella flexneri (serotype 2B) bacteria cultured in LB media containing 32 μg/mL rifaximin. A fourth culture contained Shigella flexneri (serotype 2B) bacteria in LB media containing both 32 μg/mL rifaximin and 4 mM of total bile acids. These results clearly demonstrate that the addition of bile acids to rifaximin also increases its ability to inhibit the growth and protein synthesis in Shigella flexneri, rendering previously sub-lethal concentrations of rifaximin lethal.

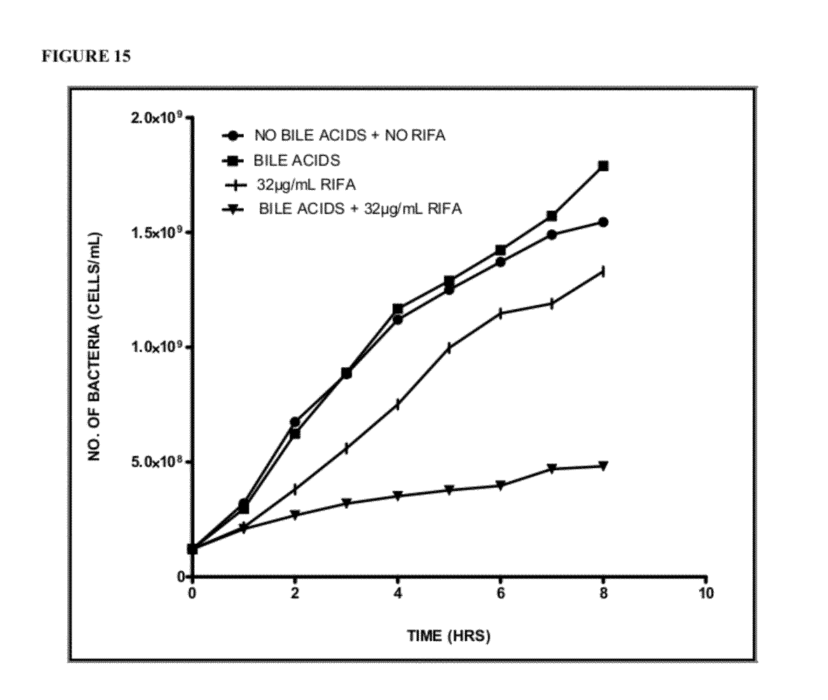

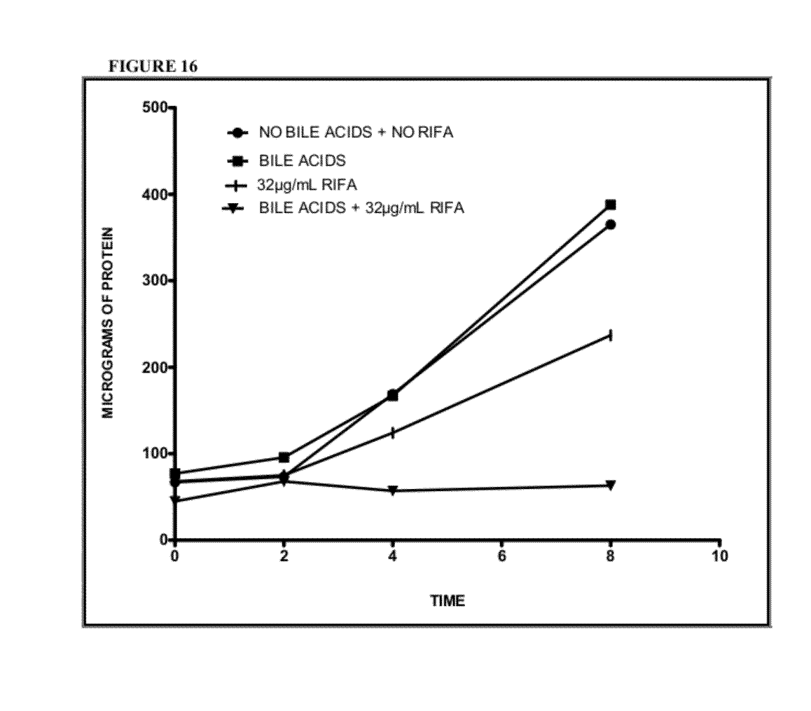

Salmonella enterica: The minimal inhibitory concentration (MIC) for the bacterial strain Salmonella enterica (ATCC #14028) for rifaximin is 64 μg/mL. Therefore, a sub-lethal concentration of rifaximin (32 μg/mL) was used to establish that the addition of bile acids also enhanced the activity of rifaximin to inhibit the growth of Salmonella enterica (ATCC #14028) as shown in FIG. 15 and protein synthesis as shown in FIG. 16.

The untreated control contained Salmonella enterica (without rifaximin or bile acids) cultured in LB media and a growth curve was determined by measuring the OD₆₀₀ at sixty minutes intervals during the incubation period (as described previously). A second culture of Salmonella enterica bacteria in LB media to which was added 4 mM of bile acids (comprising an equimolar mixture of each of the bile acids: cholic, deoxycholic, chenodeoxycholic, glycocholic, lithocholic, and taurocholic acids at pH 7.4). A third culture contained Salmonella enterica (ATCC #14028) bacteria cultured in LB media containing 32 μg/mL rifaximin. A fourth culture contained Salmonella enterica bacteria in LB media containing both 32 μg/mL rifaximin and 4 mM of total bile acids. These results clearly demonstrate that the addition of bile acids to rifaximin also increases its ability to inhibit the growth and protein synthesis in Salmonella enterica, rendering previously sub-lethal concentrations of rifaximin lethal.

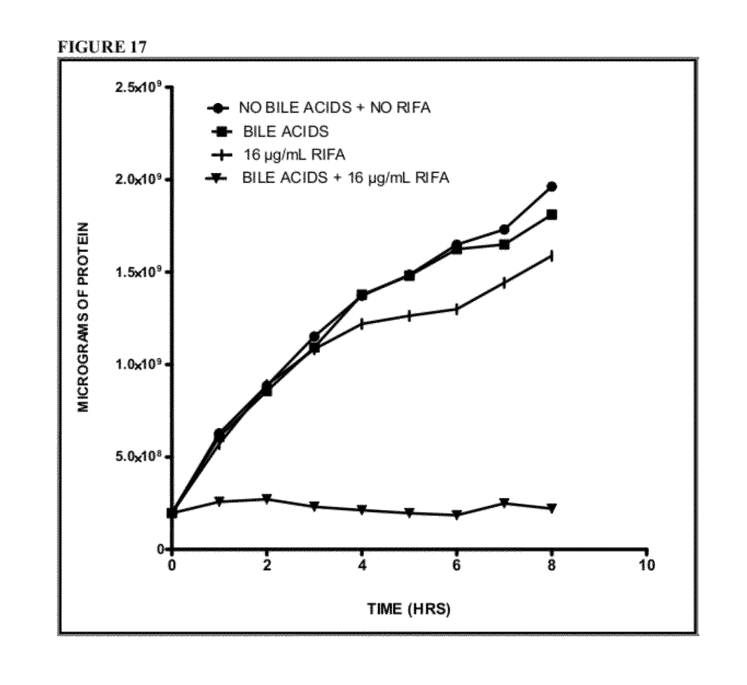

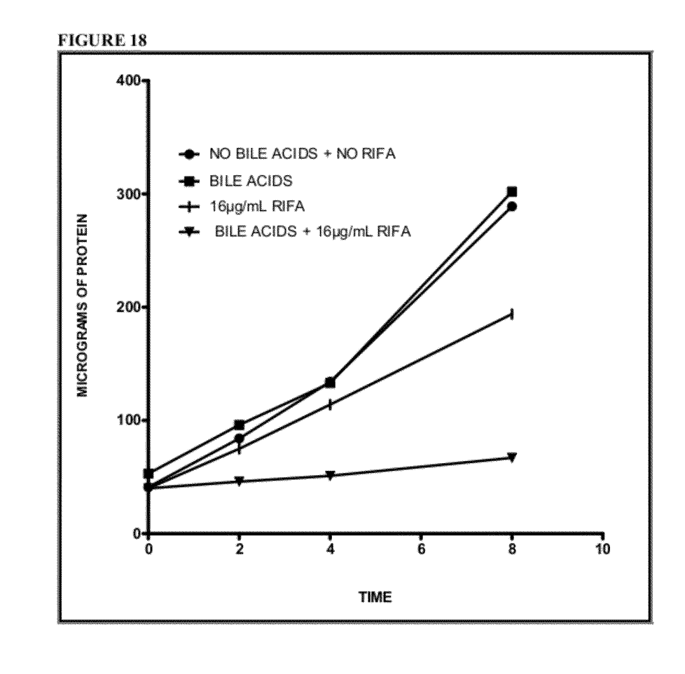

Enteroaggregative E. coli: The minimal inhibitory concentration (MIC) of rifaximin for the Enteroaggregative strain of E. coli used is 32 μg/mL. Therefore, a sub-lethal concentration of rifaximin (16 μg/mL) was used to establish that the addition of bile acids also enhanced the activity of rifaximin to inhibit the growth of Enteroaggregative E. coli as shown in FIG. 17 and protein synthesis as shown in FIG. 18.

The untreated control contained Enteroaggregative E. coli (without rifaximin or bile acids) cultured in LB media grid a growth curve was determined by measuring the OD₆₀₀ at sixty minutes intervals during the incubation period (as described previously). A second culture of Enteroaggregative E. coli bacteria in LB media to which was added 4 mM of bile acids (comprising an equimolar mixture of each of the bile acids: cholic, deoxycholic, chenodeoxycholic, glycocholic, lithocholic, and taurocholic acids at pH 7.4). A third culture contained Enteroaggregative E. coli bacteria cultured in LB media containing 16 μg/mL rifaximin. A fourth culture contained Enteroaggregative E. coli bacteria in LB media containing both 16 μg/mL rifaximin and 4 mM of total bile acids. These results clearly demonstrate that the addition of bile acids to rifaximin also increases its ability to inhibit the growth and protein synthesis in Enteroaggregative E. coli, rendering previously sub-lethal concentrations of rifaximin lethal.

In combination, the results of the studies described herein clearly indicate that the addition of bile acids increase the antibacterial activity of rifaximin. Thus, rendering what had previously been sub-lethal concentrations of rifaximin effective for the treatment of many pathogens.

Without further elaboration, it is believed that one skilled in the art can, using the description herein, utilize the present methods to its fullest extent. The embodiments described herein are to be construed as illustrative and not as constraining the remainder of the disclosure in any way whatsoever. While preferred embodiments have been shown and described, many variations and modifications thereof can be made by one skilled in the art without departing from the spirit and teachings of the presently disclosed methods. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

TABLE 1
Cell densities of E. coli H10407 cells grown with rifaximin in the presence and absence of
both synthetic acids and human bile. Data represent cell densities after 4 hours of incubation.

| CELL DENSITY (NO. OF BACTERIA/MILLILITER OF CULTURE) | | | | | |
|---|---|---|---|---|---|
| | NO BILE | | | 4 mM BILE ACIDS + | PERCENT DECREASE IN |
| | ACID + NO | 4 mM | 16 μg/mL | 16 μg/mL | CELL DENSITY ON |
| | RIFAXIMIN | BILE ACIDS | RIFAXIMIN | RIFAXIMIN | ADDITION OF BILE ACIDS |
| BILE ACIDS | (×10⁸) | (×10⁸) | (×10⁸) | (×10⁸) | TO RIFAXIMIN (%) |
| Human bile | 9.4 ± 0.69 | 12 ± 0.30 | 6.6 ± 0.50 | 1.8 ± 0.08 | 72.8 ± 0.84 |
| Pooled synthetic bile | 9.4 ± 0.69 | 9.6 ± 0.25 | 6.6 ± 0.50 | 2.0 ± 0.74 | 70.6 ± 0.48 |
| Cholic acid | 12 ± 0.16 | 11 ± 0.46 | 4.9 ± 1.0 | 0.22 ± 0.20 | 95.5 ± 0.80 |
| Deoxycholic acid | 12 ± 0.16 | 3.9 ± 0.34 | 4.9 ± 1.0 | 0.30 ± 0.49 | 93.8 ± 0.51 |
| Chenodeoxycholic acid | 12 ± 0.16 | 7.0 ± 0.06 | 4.9 ± 1.0 | 0.53 ± 0.51 | 89.2 ± 0.49 |
| Glycocholic acid | 12 ± 0.16 | 14 ± 0.32 | 4.9 ± 1.0 | 0.94 ± 0.60 | 80.8 ± 0.40 |
| Taurocholic acid | 12 ± 0.16 | 14 ± 0.51 | 4.9 ± 1.0 | 1.1 ± 0.23 | 77.6 ± 0.77 |
| Lithocholic acid | 12 ± 0.16 | 6.6 ± 0.25 | 4.9 ± 1.0 | 2.8 ± 0.27 | 43.9 ± 0.73 |

What is claimed is:

1. A composition comprising a rifamycin compound and at least one bile acid.

2. The composition of claim 1, wherein the rifamycin compound is rifaximin.

3. The composition of claim 2, wherein the rifaximin compound comprises a polymorphic form of rifaximin.

4. The composition of claim 1, wherein the at least one bile acid is selected from the group consisting of cholic acid, chenodeoxycholic acid, deoxycholic acid, lithocholic acid, glycocholic acid, taurocholic acid, glycocholic acid, glycodeoxycholic acid, glycochenodeoxycholic acid, taurocholic acid and taurodeoxycholic acid, glycolithocholic acid, taurolithocholic acid, taurohyodeoxycholic acid, taurochenodeoxycholic acid, ursocholic acid, tauroursodeoxycholic acid, and glycoursodeoxycholic acid.

5. The composition of claim 4, wherein the bile acid is cholic acid.

6. The composition of claim 1, wherein the at least one bile acid is human bile acid.

7. The composition of claim 1, wherein the at least one bile acid is synthetic bile acid.

8. The composition of claim 1, wherein the at last one bile acid forms mixed micelles in water.

9. The composition of claim 1, comprising nanoparticles containing said rifamycin compound and said one or more bile acid.

10. The composition of claim 1, wherein said composition is aqueous and the solubility of said rifaximin in said aqueous composition is enhanced compared to a like composition lacking said at least one bile acid.

11. A pharmaceutical composition comprising a rifamycin compound; at least one bile acid; and a pharmaceutically acceptable carrier.

12. The pharmaceutical composition of claim 11, wherein the at least one bile acid is a synthetic bile acid.

13. A method of treating an infection of the gastrointestinal tract or colon in a subject, comprising: administering to the subject a composition comprising a rifamycin compound and at least one bile acid.

14. A method for increasing the efficacy of rifaximin treatment of a gastrointestinal disorder in a subject, comprising:
administering to the subject the composition of claim 11, and thereby increasing the efficacy of an amount of rifaximin for treating said disorder as compared to treatment of said disorder in said subject with said amount of rifaximin in the absence of said at least one bile acid.

15. A method for increasing the solubility of rifaximin in aqueous solution, comprising:
formulating an aqueous composition comprising the composition of claim 11, wherein the solubility of said amount of rifaximin in said aqueous composition is greater than the solubility of said amount of rifaximin in an aqueous composition lacking said at least one bile acid.

16. The method of claim 15, wherein the rifaximin comprises a polymorphic form of rifaximin.

17. The method of claim 15, wherein the at least one bile acid is selected from the group consisting of cholic acid, chenodeoxycholic acid, deoxycholic acid, lithocholic acid, glycocholic acid, taurocholic acid, glycocholic acid, glycodeoxycholic acid, glycochenodeoxycholic acid, taurocholic acid and taurodeoxycholic acid, glycolithocholic acid, taurolithocholic acid, taurohyodeoxycholic acid, taurochenodeoxycholic acid, ursocholic acid, tauroursodeoxycholic acid, and glycoursodeoxycholic acid.

18. The method of claim 17, wherein the bile acid is cholic acid.

19. The method of claim 15, wherein the at least one bile acid is human bile acid.

20. The method of claim 15, wherein the at least one bile acid is synthetic bile acid.

21. The method of claim 15, wherein the at least one bile acid forms mixed micelles in aqueous mixtures.

22. The method of claim 15, wherein the composition comprises nanoparticles containing said rifamycin compound and said one or more bile acids.

23. A kit comprising;
a composition, wherein the composition comprises:
a rifamycin compound;
at least one bile acid; and
a pharmaceutically acceptable carrier; and
instructions for use.

24. The kit of claim 23, wherein the rifamycin compound is rifaximin.
